(19) United States
(12) Patent Application Publication
(10) Pub. No.: US 2025/0297212 A1
(43) Pub. Date: Sep. 25, 2025

(54) IN VITRO DETERMINATION OF CLONAL COMPOSITION IN EMBRYOS

(71) Applicants: California Institute of Technology, Pasadena, CA (US); Cambridge Enterprise Limited, Cambridge (GB)

(72) Inventors: Magdalena D. Zernicka-Goetz, Pasadena, CA (US); Maciej Meglicki, Cambridge (GB); Sergi Junyent Espinosa, Pasadena, CA (US)

(21) Appl. No.: 19/083,211

(22) Filed: Mar. 18, 2025

Related U.S. Application Data
(60) Provisional application No. 63/567,222, filed on Mar. 19, 2024.

(51) Int. Cl. C12N 5/073; G01N 33/50

(52) U.S. Cl. CPC C12N 5/0604; G01N 33/5088; C12N 2500/30; C12N 2501/998; C12N 2506/02

(57) ABSTRACT

Provided herein include compositions and in vitro methods for culturing, imaging, and determining a clonal composition in embryos. Provided herein also includes a computer-based method of determining a clonal composition in embryos models through stochastic modeling.

### RELATED APPLICATIONS

This application claims the benefit under 35 U.S.C. § 119(e) of U.S. Provisional Patent Application Ser. No. 63/567,222, filed Mar. 19, 2024, the content of this related application is incorporated herein by reference in its entirety for all purposes.

### BACKGROUND

### Field

The present disclosure relates generally to the field of embryo development and related testing, and particularly the culturing and imaging of embryos.

### Description of the Related Art

Retrospective construction of developmental cell lineages in humans predicted that most of the body is derived from just one of the two blastomeres of the 2-cell embryo. These findings implied an early bottleneck in development, but the mechanisms were unclear. There is a need to develop in vitro methods and mathematical models for lineage tracing and imaging of live human embryos.

### SUMMARY

Provided herein includes a method for determining a clonal composition of an embryo. The method can comprise, in some embodiments, culturing an embryo at the zygote stage in a first embryo culture media until the embryo forms 2-cell blastomeres; labeling one blastomere of the 2-cell blastomeres with a detectable lineage marker; culturing the 2-cell blastomeres in a second embryo culture media for about 4 to 5 days allowing the 2-cell blastomeres to develop into a blastocyst; detecting cells expressing the detectable lineage marker in the blastocyst; and quantifying the clonal composition of the inner cell mass (ICM) and trophectoderm (TE) based on the detection of cells expressing the detectable lineage marker.

The first embryo culture media and the second embryo culture media can be the same or different. In some embodiments, the first embryo culture media and/or the second embryo culture media comprises amino acids, physiological salts, a carbon source, an antibiotic, and a buffer, optionally, the carbon source is glucose. The antibiotic can comprise, for example, Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The first embryo culture media and/or the second embryo culture media can comprise, for example, sodium chloride, potassium chloride, calcium chloride potassium phosphate, magnesium sulfate, sodium bicarbonate, glucose, sodium lactate, sodium pyruvate, amino acids, EDTA and gentamicin sulfate. In some embodiments, the first embryo culture media and/or the second embryo culture media is substantially protein-free. In some embodiments, the first embryo culture media and/or the second embryo culture media further comprises non-human serum or serum substitute. In some embodiments, the non-human serum or serum substitute comprises fetal bovine serum, bovine serum albumin, human serum albumin, or any combination thereof. In some embodiments, first embryo culture media and/or the second embryo culture media comprises human α- and β-globulins.

In some embodiments, the embryo at the zygote stage is cultured in the first embryo culture media for about 12-20 hours until the completion of the first cleavage division. In some embodiments, the detectable lineage marker does not affect the development of the embryo to the blastocyste stage and enables annotation of the position and boundaries of cells in the embryo. In some embodiments, labeling the one blastomere of the 2-cell blastomeres with the detectable lineage marker comprises injecting the blastomere with an mRNA encoding the detectable lineage marker. The blastocyst can be, e.g., a non-expanded blastocyst or an expanded blastocyst.

In some embodiments, the method comprises selecting a subset of embryos at 4-cell stage, 8-cell stage, 16-cell stage, and/or 32-cell stage from the second embryo culture media prior to the formation of the blastocyst, and live staining the subset of embryos. In some embodiments, live staining the subset of embryos comprises culturing the selected subset of embryos in an embryo culture media containing dyes. The dyes can be, e.g., membrane-permeable fluorescent dyes capable of tracking both genomic nucleic acids and a component of cytoskeleton of the embryos.

In some embodiments, the selected subset of embryo is cultured in the embryo culture media containing dyes for about 25-28 hours. In some embodiments, the method comprises monitoring asymmetric cell division events at 2- to 4-cell transition, 4- to 8-cell transition and/or from 8- to 16-cell transition. In some embodiments, monitoring asymmetric cell division events comprises counting the number of asymmetric cell division events or the number of cell internalizations at 2- to 4-cell transition, 4- to 8-cell transition and/or from 8- to 16-cell transition. In some embodiments, the clonal composition of the ICM and TE in the blastocyst is quantified based on the contribution of cells expressing the detectable lineage marker and cells not expressing the detectable lineage marker in the ICM and TE of the blastocyst. In some embodiments, the quantification comprises determining the number, size and/or position of cells expressing the detectable lineage marker and cells not expressing the detectable lineage marker in the ICM and/or the TE of the blastocyst. In some embodiments, quantifying the clonal composition of the ICM and TE further comprises identifying the dominant clonal composition in the ICM and/or the TE of the blastocyst. In some embodiments, quantifying the clonal composition of the ICM and TE further comprises determining the percentage of cells expressing the detectable lineage marker and/or cells not expressing the detectable lineage marker in the ICM and/or TE of the blastocyst.

In some embodiments, the method comprises assigning a score to the blastocyst based on the blastocyst development stage status, the inner cell mass number and quality, and/or the trophectoderm cell number and quality. In some embodiments, the method comprises performing blastocyst ploidy analysis. As described herein, the embryo can be a human embryo.

Provided herein includes a method of selecting embryos. The method can comprises, in some embodiments, providing a plurality of embryos at the zygote stage, determining a clonal composition of each embryo of the plurality of embryos according to any one of claims 1-25, and selecting embryos having a desired clonal composition based on the percentage of cells expressing the detectable lineage marker and/or cells not expressing the detectable lineage marker in the inner cell mass (ICM) and trophectoderm (TE) of an embryo at the blastocyst stage. In some embodiments, the selected embryo comprises clonally imbalanced ICM. In some embodiments, the ICM of the selected embryos is clonally symmetric. In some embodiments, the embryos are human embryos.

Provided herein includes a computer-based method of determining a clonal composition in embryo models. The method can comprises, in some embodiments, (i) generating a plurality of embryo models each comprising two cells, wherein one cell of each embryo model is randomly marked; (ii) modulating a set of parameters comprising a cell death rate, a cell arrest rate, and a number of asymmetric cell divisions for a stochastic model; (iii) subjecting the plurality of embryo models to the stochastic model wherein each embryo model undergoes successive rounds of cell division until the embryo model reaches a desired total number of cells; and (iv) determining a clonal composition of the inner cell mass (ICM) and trophectoderm (TE) for each embryo model reaching the desired total number of cells. In some embodiments, the method comprises repeating steps (i)-(iv) for at least 10, 100, 1000, 10000, 100000, 1000000 or more times. In some embodiments, the successive rounds of cell division comprise a 2- to 4-cell transition, a 4- to 8-cell transition, a 8- to 16-cell transition, a 16- to 32-cell transition, and/or a 32- to 64-cell transition. In some embodiments, each embryo model undergoes at least five rounds of cell division. In some embodiments, the desired total number of cells is at least 64. In some embodiments, modulating the set of parameters comprises selecting the number of asymmetric cell divisions for the 8- to 16-cell transition, 16- to 32-cell transition, and/or 32- to 64-cell transition, optionally, the number of asymmetric cell divisions is selected as 0, 1, 2, or 3. In some embodiments, the number of asymmetric cell divisions for the 8- to 16-cell transition is selected as 1, 2 or 3, the number of asymmetric cell divisions for the 16- to 32-cell transition is selected as 1 or 2, and/or the number of asymmetric cell divisions for the 32- to 64-cell transition is selected as 0 or 1. In some embodiments, the number of asymmetric cell division prior to the 8-cell stage is selected as zero and/or the number of asymmetric cell division after the 64-cell stage is selected as zero.

In some embodiments, marked cells and unmarked cells in an embryo model have equal or substantially equal probability for an asymmetric cell division. In some embodiments, marked cells and unmarked cells in an embryo model has unequal probability for an asymmetric cell division. In some embodiments, the marked cells or the unmarked cells have a fate bias determination rate of about 0.5 to about 0.8.

In some embodiments, modulating the set of parameters comprises selecting the cell death rate for cell divisions beyond the 64-cell stage. In some embodiments, the cell death rate is selected such that the average percentage of dead cells at the blastocyst stage is in the range of 7-8%. In some embodiments, modulating the set of parameters comprises selecting the cell arrest rate at the 4-cell stage and/or the 8-cell stage. In some embodiments, the cell arrest rate is selected as a value of about 6.5%. In some embodiments, modulating the set of parameters comprises fitting the set of parameters to in vitro clonal composition data.

In some embodiments, the method comprises providing the in vitro clonal composition data. In some embodiments, the in vitro clonal composition data is obtained from one or more methods described herein. In some embodiments, the determined clonal composition comprises the percentage of marked cells and/or unmarked cells in the ICM and/or TE of each embryo model. In some embodiments, the plurality of embryo models is a plurality of human embryo models.

Also provided herein includes a method for investigating the effect of a test agent on embryonic development. The method can comprise, in some embodiments, contacting a test agent with an embryo at the zygote stage; determining a clonal composition of the embryo using one or more methods describe herein; and determining the effect of the test agent on the clonal composition, and optionally the determining comprises comparing the clonal composition obtained in the presence of the test agent with a clonal composition obtained in the absence of the test agent. The embryo can be a human embryo

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1L depict non-limiting exemplary data related to lineage tracing of 2-cell stage human blastomeres in the blastocyst. FIG. 1A is a schematic depicting the pipeline of the experiment. FIG. 1B shows an example of human blastocyst with mosaic expression of GFP, stained with AF647-Phalloidin (orange) and DAPI (blue). Scale bar=50 μm. FIG. 1C is a plot showing frequency of GFP+ and GFP− cells in the whole embryo, for each embryo quantified. FIG. 1D is plots showing percentage of GFP+ cells in the whole embryo (grey), TE (red) or ICM (blue) (left panel) and contribution of GFP+ cells in the whole embryo, TE or ICM for each embryo, connected (right panel). FIG. 1E depicts a schematic of terminology used. The population (GFP+ or GFP−) that contributed >50% to each compartment (ICM or TE) was considered dominant for that compartment. In some cases, the ICM-dominant population was used as reference, and the matching population in the TE was called “ICM-dominant population in the TE”. FIG. 1F is a chart showing contribution of dominant (D) or non-dominant (ND) cells to TE (red) and ICM (cyan). Statistically significant difference (ICM vs TE) of differences (Dominant vs non-dominant) assessed by t-test. FIG. 1G shows representative images of human blastocysts with different degrees of clonal imbalances in ICM and TE. Spots label the cell centers, colored for TE (red), ICM (cyan), GFP+ (green) or GFP− (grey). Contribution of ICM-dominant cells to each compartment displayed in figure. Scale bar=50 μm. FIG. 1H is a graph showing contribution of ICM-dominant cells to ICM and TE. Marginal histograms indicate distribution. Grey areas represent contribution 50-60% to ICM and 40-60% to TE. FIG. 1I is a graph plotting contribution of ICM-dominant cells to TE (red) or ICM (cyan). Statistically significant differences assessed by t-test. FIG. 1J depicts a schematic of SOX17 staining differentiating epiblast (EPI) and hypoblast (HYPO) in ICM. FIG. 1K is a graph showing contribution of EPI-dominant population to EPI or HYPO. Statistically significant differences assessed by t-test. Two blastocysts from the pilot experiment were not included in the analysis because they were not accessible for SOX17 staining (n=20 human blastocysts). FIG. 1L are images showing maximum intensity projection (MIP) (left) or computerized reconstruction (right) of a mostly GFP+ ICM containing GFP+ and GFP− hypoblast cells. Scale bar=25 μm. For C, D, G and I, n=22 blastocysts. For F, I and K, error bars are 10-90 percentile, box is upper and lower quartiles, center line is median.

FIGS. 2A-2K depict non-limiting exemplary data demonstrating that the ICM-dominant blastomere is enriched in the polar TE. FIG. 2A is an image showing the quantification performed in B-E. 3D blastocyst expressing GFP and stained with DAPI. Dotted white line delimits position of GFP− cells in TE. Reconstructions show shape and position of GFP+ (green) and GFP− (grey) clusters. Scale bar=50 μm. FIG. 2B is a graph showing the number of GFP+ (green) or GFP− (grey) cell clusters/embryo in TE. FIG. 2C is a graph showing the number (top) or percentage (bottom) of cells that compose each cell cluster, per embryo. Green: GFP+ clusters; grey: GFP− clusters. Each cluster (1-8) is ordered by size, (cluster 1=largest). Each cluster is shown by diminishing tones color. FIG. 2D are graphs showing (Left) Orientation of embryonic-abembryonic axis in a blastocyst. (Middle) Area covered by ICM in each embryo (blue). (Right) Border separating the largest GFP+ and GFP− cell clusters in TE. For most embryos, the border separating the clonal cell clusters runs through the embryonic-abembryonic axis. FIG. 2E is a graph showing presence of the largest GFP+ and GFP− clusters in polar. Values indicate number of embryos. FIG. 2F is a schematic depicting quantifications performed in G-I. FIGS. 2G-2I are plots showing contribution of the ICM-dominant population to ICM versus total TE (red, FIG. 2G), polar TE (yellow, FIG. 2H) or mural TE (orange, FIG. 2I). R² and p-value against a line with a zero slope displayed in the figure. FIG. 2J are images showing example human blastocysts expressing GFP (green) and stained with DAPI (blue) and AF647-Phalloidin (orange), corresponding to quantifications shown in H. ICM and pTE magnifications are displayed in each panel as fluorescence image (top) or computerized reconstruction (bottom). Scale bar=50 μm. FIG. 2K are images showing computerized reconstructions of ICM (blue) and polar TE (orange). Dots are cells, colored according to GFP expression. Top and side views are presented. Scale bars=20 μm. For FIGS. 2B-2D and 2G-2I, n=21 human blastocysts. One embryo was excluded from the analysis as it was damaged during processing and TE spatial composition could not be assessed.

FIGS. 3A-3J depict non-limiting exemplary data demonstrating that 2-cell clonal imbalance is not explained by blastomere arrest or genomic instability. FIG. 3A are images showing human blastocyst with an arrested blastomere on TE surface. Blastocysts mosaic for GFP stained with AF647-Phalloidin (orange) and DAPI (blue). (Right) Reconstructions of TE (red), ICM (blue) and arrested blastomere (green). Scale bar=50 μm. FIG. 3B is a graph showing number of arrested blastomeres measured (n=22 blastocysts). Side bars colored by degree of clonal imbalance in ICM or TE. FIG. 3C is a graph showing the IVF embryos percentage. n=88 embryos analyzed. FIG. 3D are images showing quantification and representative frames of a time-course imaging depicting the emergence of an arrested blastomere in an 8-cell stage human embryo. Scale bar=50 μm. For FIGS. 3B and 3C, numbers indicate the embryo number. FIG. 3E are images showing human blastocyst with a cell containing cytoDNA. Red arrowhead indicates cytoDNA. Scale bar=50 μm. FIGS. 3F-3H are plots showing proportion of cells with cytoDNA in the whole embryo (FIG. 3F), GFP+ or GFP− cells (FIG. 3G), TE or ICM (FIG. 3H). For FIG. 3H, dots are colored according to the degree of clonal imbalance in ICM or TE. n=22 human blastocysts. Center lines are mean, error bars are standard error. Statistical significance assessed by t-test. FIG. 3I is a schematic depicting the protocol. FIG. 3J is a summary table reporting the results of the sequencing (n=20 embryos). Two embryos could not be included in the analysis due to unavailability of the material for the ploidy analysis.

FIGS. 4A-4F depict non-limiting exemplary data related to non-invasive labelling of human embryos. FIG. 4A is images showing dividing mouse 4-cell stage blastomere stained with SPY555-DNA and SiR-Actin. Scale bar=50 μm. FIG. 4B is plots showing (Left) Cell cycle length (min) in mouse embryos (8-cell to 16-cell stage) stained with SPY555-DNA and Sir-Actin, or not stained (N=32 cells from n=4 embryos). (Middle) Interphase length (time between end of one division and beginning of the next mitosis; min) in mouse and human embryos during 8-cell to 16-cell transition. (Right) Mitosis length (time between prophase and end of anaphase; min) in mouse and human embryos during the 8-cell to 16-cell transition. For all plots, center line is mean, error bars are standard error. For B statistical significance assessed by t-test. Mouse quantification (N=42 cells from n=6 embryos), human quantification (N=24 cells from n=4 embryos). FIG. 4C shows representative images and quantification of the apical F-actin ring progression in 8-cell stage mouse blastomeres, stained with SPY555-DNA and SiR-Actin. Scale bar=50 μm, highlighted band is quantification profile, white arrowheads indicate edges of apical ring. FIG. 4D is images showing Human live embryos expressing mGap43-GFP (green) in some cells, stained with SiR-Actin (red) and SPY555-DNA (yellow), and imaged at different timepoints. Scale bar=50 μm. The embryos displayed at the 2-cell and 4-cell stage were part of the preliminary tests of the protocol, and are not the 4 embryos included in FIG. 5. FIG. 4E shows representative images of GFP− (top) and GFP+ (bottom) human blastomeres dividing. Scale bar=50 μm. FIG. 4F shows representative frames and quantification of the polarization process in human embryos. SiR-Actin (red) intensity in the membrane quantified across time. Grey thin lines are quantifications of the cortex of each cell in the embryo displayed, thick red line is average. In FIGS. 4A, 4D, 4E and 4F, zona pellucida was digitally removed during image processing.

FIGS. 5A-5G depict non-limiting exemplary data related to live imaging of human embryos revealing that asymmetric cell division at the 8-cell stage predicts ICM clonal composition. FIG. 5A is images showing symmetric (inside-outside, top panel, blue) and asymmetric (outside-outside, bottom panel, red) cell divisions in the same human embryo during the 8- to 16-cell transition. Embryos mosaic for GFP were stained with SiR-Actin and SPY555-DNA at the 8-cell stage and imaged over time. Zona was digitally removed for clarity. Scale bars=50 μm. FIG. 5B is a schematic depicting the imaging of human embryos using membrane permeable dyes. Blue and read arrowheads indicate asymmetric and symmetric cell divisions respectively. FIG. 5C is graphs showing proportion (left) and vectorial angle (right) of the asymmetric and symmetric divisions detected in the time course movies. In right, error bars are 10-90 percentile, box is upper and lower quartiles, center line is median. Statistical significance assessed by t-test. N=28 cells from n=4 human embryos. Two cells (one GFP+ and one GFP−) in Embryo #1, and one cell (GFP−) in Embryo #2 did not divide within the imaged time. Values are absolute number of cells. FIG. 5D is a graph showing number and type (GFP+ or GFP−) of cells that have asymmetric or symmetric divisions, or that do not divide. FIG. 5E is a graph showing percentage of GFP+ cells in ICM (cyan) or TE (red) at the blastocyst stage. n=4 human embryos. FIGS. 5F-5G show an exemplary embryo tracked from the 8- to 18-cell stage. Scale bar, 50 μm.

FIGS. 6A-6I depict non-limiting exemplary results related to a computational model of three parameters that predict the clonal imbalance in human blastocysts. FIG. 6A depicts a schematic representation of the model. p_{death} is death rate (red crossed cells, restricted to 64-cell onwards and ICM only); pₐᵣᵣₑₛₜ is arrest rate (magenta highlighted cell, restricted to 4- and 8-cell stages); nᵢₙₜₑᵣ is number of cells internalizing in each division generation from 8- to 64-cell stages (cyan outline, drawn uniformly from the ranges [1,3], [1,2], and [0,1] for the cell stage transitions 8→16, 16→32, 32→64, respectively, and zero earlier or later). nᵢₙₜₑᵣ can be modulated to include a bias for ACD by one clone. FIG. 6B is a graph showing observed (blue) and statistically predicted (unbiased, red; biased, yellow) distributions of GFP+ cells in 22 whole blastocysts. Model predictions in FIGS. 6B, 6F, 6H and 6I are means±SD from 10⁴ statistically independent realizations of 22 blastocysts. Inputted random cell arrest as reported in FIG. 13, panel B (6.5%), and random cell death in ICM with probability 4.4% from the 64-cell stage. All P-values in FIG. 6B (evaluated directly from the fraction of 10⁴ model predictions equally or more extreme than the observation) are >0.05 (ns) or <0.05 (*). FIG. 6C is a graph showing distributions of GFP+ cells as in FIG. 6B, by compartment. In FIGS. 6C and 6D error bars are 10-90 percentile, box is upper and lower quartiles, center line is median. All P-values in FIGS. 6C-F are from two-sample Kolmogorov-Smirnov tests. FIG. 6D is a graph showing relative size of the ICM as percent of embryo cells. FIG. 6E is a plot showing ICM-dominant clonal contributions in TE and ICM. Gray dashed lines and small gray numbers indicate the percentage of embryos with ICM-dominant contributions in the ICM higher than 60% or 80% in the “unbiased” model. FIG. 6F is a graph showing percentages of embryos with ICM-dominant contribution ≥60% in the ICM, or ≥60%-≤40% in the TE. FIG. 6G is a plot showing overall degree of statistical consistency between data and model, measured by the harmonic mean of four P-values from FIGS. 6C (whole embryo), 6D, 6E and 6F, as a function of the 2-cell clonal fate bias to become ICM. At about 80:20 bias, the P^{o}=0.05 threshold is crossed. A 70:30 bias was chosen to represent the biased case (yellow) in FIGS. 6B-F, 6H and 6I, as it is statistically representative without being a statistical edge case. FIGS. 6H-6I are P-P plots showing the predicted fraction of ICM-dominant cells in TE and ICM against the observed fraction. Each data point represents one of the 22 blastocysts with statistical mean values μᵢ and SDs σᵢ as error bars. The data do not deviate significantly from a diagonal line except for the biased case (yellow) in the ICM. R² is the coefficient of determination.

$${χ}_{red}^{2} = \frac{1}{22} {\sum }_{i = 1}^{22} {( \frac{{x}_{i} - {μ}_{i}}{{σ}_{i}} )}^{2}$$

is the reduced chi-squared test statistic, which is low in absence of statistical disagreement.

FIGS. 7A-7P depict non-limiting exemplary results related to lineage tracing revealing that faster dividing blastomere in the 2-cell human embryo is biased towards the first ACD at the 8-cell stage. FIG. 7A is a schematic depicting the pipeline of the analysis. FIG. 7B shows representative examples of an asymmetric (inside-outside, left, blue) and a symmetric (outside-outside, right panel, red) cell division in the same human embryo during the 8- to 16-cell transition, tracked in multifocal Embryoscope™ transmitted light movie. t: absolute time provided with the embryoscope movie. FIG. 7C is a graph showing percentage of human embryos with different numbers of ACDs during 8- to 16-cell division. n=54 human embryos. FIG. 7D is a graph showing percentage of embryos with 2 ACDs occurring from the same or different 2-cell clones within the embryo during 8- to 16-cell division. Observed value was compared to the value expected by combinatorial chance (see methods) using Chi-square test. n=25 human embryos. FIG. 7E is a graph showing percentage of embryos with 3 ACDs occurring from the same or different 2-cell clones within the embryo during 8- to 16-cell division. Observed value was compared to value expected by combinatorial chance (see methods) using Chi-square test. n=12 human embryos. FIG. 7F is a graph showing percentage of 16-cell stage human embryos with different levels of clonal dominance in iCM. n=54 embryos. FIG. 7G is a graph showing correlation between clonal dominance in ICM at 16-cell stage and clonal dominance in ICM at blastocyst stage. Data was extracted from the model presented in FIGS. 6A-6I. Modelled embryos with clonal compositions matching the ones reported in FIG. 7F were chosen, and their ICM composition at the blastocyst stage was recorded. Red boxes indicate unbiased model, yellow boxes indicate model with a clonal fate bias to become ICM at 70%. Error bars are 10-90 percentile, box is upper and lower quartiles, center line is median. Pearson's r, Kendall's τ, and Spearman's ρ are specified. The lines are linear least-squares fits. FIG. 7H is a graph showing the number of human embryos with different degree of asynchrony at the 2- to 4-cell stage division. n=54 human embryos. Color indicates the number of ACD per embryo: blue (1), red (2), green (3), orange (4). FIG. 7I is a graph showing the classification of 2-cell stage blastomeres as fast (A) or slow (B) according to their order of division at the 2-to-4-cell stage. Progeny of the “A” blastomere also divides faster at the 4- and 8-cell stages (red dashed arrows). FIGS. 7J-K are plots showing percent of cells dividing in 1^{st}-4^{th} position with A (Aa or Ab) identity during 4- to 8-cell division (FIG. 7J) or percent of cells dividing in 1^{st}-8^{th} position with A (Aa1 or 2, or Ab1 or 2) identity during 8- to 16-cell division, in embryos with different degrees of asynchrony at the 2-cell stage. n=46 human embryos. In FIGS. 7J-P, synchronous embryos (n=8) were excluded as A and B blastomeres could not be identified. FIG. 7L is a graph showing number and identity of ACDs during 8- to 16-cell division originating from the faster (A) or slower (B) clone. A, orange; B, green. FIG. 7M is a graph showing percentage of embryos with first ACD coming either from A or B clone. n=46 human embryos. Dashed line indicates 50% of embryos. FIG. 7N is a graph showing percentage of embryos with ACD by the A or B blastomere. Embryos were separated by their total number of ACDs (1, n=14; 2, n=21; or 3, n=10). For embryos with 2 or 3 ACD, quantification is broken down by the order of division. FIG. 7O is a graph showing percentage of embryos at 16-cell stage containing either more inner cells from A or B, or with the same number of inner cells coming from each clone. Observed value was compared to value expected by combinatorial chance and compared using a binomial test for [A=B] vs ![A=B] cases. FIG. 7P is a schematic representation of the results in FIGS. 7L-7O. For all panels, numbers in columns indicate the embryo number. For K, L, N and O n=46 human embryos.

FIG. 8 depicts non-limiting exemplary results on the quantification of the clonal imbalances of human blastocysts related to FIGS. 1A-1L. Panel A. Breakdown of the embryos used for this project, including the number of embryos that survived thawing, that were microinjected, that developed to blastocysts and that were included in the final quantification. Rules for inclusion/exclusion are listed in the Methods. Panel B. Representative example of a human blastocyst with mosaic expression of GFP (same embryo as shown in FIG. 1B) with only DAPI staining (blue) and GFP (green) shown. Scale bar=50 μm. Panels C-E. Qualification of the blastocysts included in the analysis following the Gardner and Schoolcraft blastocyst grading system. (C) Percentage of embryos that were mid-late or late blastocysts at the time of fixation. (D and E) Percentage and number of embryos that were qualified as in “Good”, “Fair” or “Poor” fitness at the blastocyst stage (details in the Methods). n=22 human embryos. In E. Embryos are colored according to their degree of clonal imbalance. Panel F. Quantification of the number of total cells (grey), trophectoderm cells (red) and inner cell mass cells (ICM, blue) in the blastocyst images. Error bars are 10-90 percentile, box is upper and lower quartiles, center line is median. n=22 human embryos. Panel G. Representative maximum intensity projection (MIP), showing the expression of SOX17 in a subset of cells of the ICM (indicated by white dashed line). Embryos expressed mosaic GFP and were stained for AF647-Phalloidin (orange) and DAPI (blue). Scale bar=50 μm. Panel H. Quantification of the number of epiblast cells (cyan) and hypoblast cells (purple) in the blastocyst images. Error bars are 10-90 percentile, box is upper and lower quartiles, center line is median. n=20 human embryos. For H and I, two embryos from test experiment were not included in the analysis because they were not accessible for SOX17 staining. Panel I. Scatter plot indicating the correlation between the number of hypoblast cells per embryo versus the number of cells in the ICM for that embryo. Trend line indicates linear regression. R² and p-value against a line with zero slope are displayed in the figure. n=20 human embryos. Panel J. Scatter plot presenting the percentage of GFP+ cells in the TE and ICM of each of the embryos analyzed. n=22. Gray bands indicate 40-60% windows (low clonal imbalance). Marginal histograms report the counts. Panel K. Plot presenting the percentage of embryos for which GFP+ (green) cells or GFP− (grey) cells are dominant in the whole embryo. The dominant population is defined as the population that contributes >50% of cells in the embryo. n=22 human blastocysts. Panel L. Plot presenting the percentage of embryos that have matching (dark gray) or non-matching (light gray) populations in the TE and ICM. In matching embryos, the same 2-cell clone is the dominant (>50% of cells) population in the ICM and the TE. Side bar indicates, with colors, the degree of clonal imbalance of each individual embryo contained in the previous quantification. n=22 human blastocysts. Panel M. Scatter plot displaying the ICM size counted in absolute number of cells in relation to ICM dominant clone contribution to the ICM. n=22 human blastocysts. Panels N-P. Bar plots depicting the number of GFP+ and GFP− cells in the whole embryo (N), the TE (O) and the ICM (P) for each embryo analyzed. Boxes underneath indicate the degree of clonal imbalance in the ICM or TE of each embryo. n=22 human blastocysts. For all applicable panels, numbers in columns indicate the embryo number.

FIG. 9 shows non-limiting exemplary results related to computerized reconstructions of the position of GFP+ and GFP− cells in the human blastocysts. Spots indicate cell centers, and are colored according to GFP expression and position (ICM or TE) as indicated in the legend. Insets show the quality score classification (QS), percentage of either GFP+ or GFP− cells in the ICM, polar TE (pTE) or mural TE (mTE), and a circular plot depicting the area occupied by the ICM and the TE area covered by the largest GFP+ and GFP− cell cluster. Yellow frame indicates embryos in which the TE clonal border crosses the ICM. One embryo was excluded as it was damaged during sample processing, which precluded us to perform this analysis.

FIG. 10 is plots presenting the DNA sequencing results for the embryos presented in FIG. 3J. For two embryos (#2 and #10), the embryonic part sequencing results were designated as unreliable by a technical expert. For all plots, orange line set at 2 indicates euploidy (n2 diploid chromosome count), monosomies (n1) are indicated with a red line, and trisomies (n3) are indicated with a blue line. Chromosomes are labelled in the X axis. Two embryos could not be included in the analysis.

FIG. 11 depicts non-exemplary results from staining of human and mouse embryos with SiR-actin and SPY555-DNA. Panel A. Representative frames of a time course imaging of an example of a human embryo with lagging chromosome at the 4-cell stage. Embryos mosaic for GFP were stained with SiR-Actin and SPY555-DNA, and imaged. Dashed white box is the reconstruction of the SPY555-DNA signal, indicating a lagging chromosome (red arrowhead), and the presence of a cytoplasmic DNA (cytoDNA). This embryo arrested and degenerated before reaching blastocyst stage and thus was not included in the final analysis. Zona pellucida was digitally removed during image processing. Scale bars=10 or 25 μm, as stated in the figure. Panel B. Representative frames depicting a mouse embryo stained with SiR-Actin and developing until the blastocyst stage. Scale bar=50 μm. Panel C. Percentage of mouse embryos stained with SiR-Actin that develop to the blastocyst. N=17 embryos imaged from the 2-cell stage.

FIG. 12 depicts non-limiting exemplary results from lineage tracing of 8- to 16-cell transitions in human embryos. Panels A-D. Details corresponding to the cell tracking on the four embryos shown in FIGS. 5A-5G. (Left) Trees generated by lineage tracking: Branches indicate divisions, spots indicate timepoints, bottom squares indicate the final position of the cell (outside: red, inside: blue). Grey squares on the side indicate final ICM and TE composition quantified at the blastocyst stage for those embryos. (Right) Reconstructions of the embryos analyzed for the first (t=0 h) and last (t=27 h) timepoint. GFP+ cells are reconstructed in green, GFP− are grey. Annotations in the images refer to the lineage trees. Numbers with an apostrophe indicate daughter cells from the same division. The last panel displays the ball and chain representation of the cell movement over time. Scale bars=50 μm.

FIG. 13 depicts non-limiting exemplary results related to additional modeling parameters. Panel A. Percentage of dead cells in the blastocyst as a function of the death rate (p_{death}) applied from the 64-cell stage onwards. A p_{death} of 4.4% results in 7-8% dead cells in the blastocyst. Panel B. Dependency of the statistical consistency on the cell arrest rate applied at the 4- and 8-cell stage. Harmonic mean P^{o} (as defined in FIG. 6) is maximal at an arrest rate of 6.5%. Panel C. Dependency of blastocyst development on the timepoint of cell arrest. Arresting cells exclusively at single cell stages with the probability pₐᵣᵣₑₛₜ (top) required for the correct percentage of embryos with arrested blastomere(s) as in Panel D, leads to mild reduction in overall statistical agreement between model and data (bottom). Early arrest is statistically favored. In the bottom, colors indicate results using the unbiased (red) or 70% biased (yellow) model. Panel D. Proportion of actual (blue) or modelled embryos with at least one arrested blastomere in an unbiased (red) or 70% biased (yellow) model. The arrest rate calculated in B was applied at the 4- or 8-cell stages. Bars are mean, error bars are SD. Two-sided P-value evaluated directly as the probability of observing a value at least as extreme in the 220,000 modelled embryos. Panel E. Heat plot indicating the modelled size of the ICM using the unbiased model when the number of internalized cells (nᵢₙₜₑᵣ) in the first (8 to 16-cell), second (16 to 32-cell) or third (32 to 64-cell) wave of ACD is modulated. Box color indicates ICM size as percentage of blastocyst cells. Black star indicates the size calculated from the labelled embryos (17.4%). Panel F. Robustness of blastocyst development to changes in the timepoint of cell internalization. Internalizing cells exclusively at single cell stages in numbers nᵢₙₜₑᵣ (top) required for the correct ICM size as in FIG. 1D, largely preserves the overall statistical agreement between model and data (bottom) only in the unbiased case. Later internalization requires stronger fate bias to explain the observed data. Forced early internalization makes a bias less likely. Panel G. Statistical (dis)agreement between observed and modelled embryos under perturbation of the internalization numbers nᵢₙₜₑᵣ and modelled fate bias. When many cells internalize in the same wave (blue), the observed clonal composition in the TE and ICM are best reproduced by a model with 65-70% fate bias, as otherwise the ICM composition is too balanced. Internalizations spread more uniformly across waves (red) make a fate bias less likely. Very few ACD in the first two waves are generally unlikely.

FIG. 14 depicts non-limiting exemplary results from Embryoscope™ lineage tracing. Panel A. Representative frames from multifocal Embryoscope™ transmitted light time-lapse movies used in the study, covering preimplantation development of human embryos from zygote to blastocyst stage. Representative single focal planes and time points were selected for the purpose of this example. t—absolute time included in the embryoscope movie. Panel B. Representative example of an asymmetric (inside-outside) cell division analysis presented on main FIG. 7B, tracked in multifocal Embryoscope™ transmitted light time-lapse movie. Five different focal planes (Z) are presented. Top panel shows timepoint (t=66.3 h) before asymmetric cell division and bottom panel shows a timepoint (66.5 h) after one of the blastomeres has divided asymmetrically. Cell dividing asymmetrically is segmented in grey, cells at the bottom and top of the embryo are segmented yellow and red, respectively. Segmented cells are reconstructed on the right. t-absolute time provided with the embryoscope movie. Yellow box highlights the plane presented in FIG. 7B. Panel C. Developing the embryos with their exact clonal compositions from the Embryoscope™ dataset from the 16-cell stage to the blastocyst stage with the statistical model results in distributions in the TE and ICM (red & yellow) that are statistically compatible with those observed in the labelled embryos (blue, as in FIG. 6E). Panel D. Quantification of the percentage of human embryos with different number of ACDs (asymmetric cell divisions) during 8- to 16-cell division, and with different patterns and order of faster (A) and slower (B) 2-cell clone contribution to ACDs. Blastomere identity (A) was assigned as given in FIG. 7I. n=46 human embryos.

FIG. 15 depicts non-limiting exemplary results from validation of the model against dataset of 32-cell stage mouse embryo composition from Bischoff et al., 2008 (Development 135, 953-962. 10.1242/dev.014316). Panel A. Comparison of observed (blue) and statistically predicted (unbiased, pink) distributions of “1st clone” progenies in 66 whole blastocysts, showing few statistically significant deviations. “1^{st} clone” was the nomenclature used in the original publication to label 1 2-cell blastomere. Model predictions in A and E are means±SD from 10⁴ statistically independent realizations of the 68 blastocysts. All P-values in B (evaluated directly from the fraction of 10⁴ model predictions equally or more extreme than the observation) are >0.05 (ns), <0.05 (*), <0.01 (**), <0.001 (***) or <0.0001 (****). Panel B. Distributions of “1st clone” cells as in A, by tissue, showing no statistically significant deviations between actual data and model. In panels B and C error bars are 10-90 percentile, box is upper and lower quartiles, center line is median. All P-values in panels B-D are from two-sample Kolmogorov-Smirnov tests. Panel C. Relative size of the ICM, showing no statistically significant deviations between data and model. Panel D. “1st clone” contributions in the TE and ICM, showing no statistically significant deviations between data and model. Panel E. Percentages of embryos with a “1^{st} clone” contribution ≥60% in the ICM, or ≥60% or ≤40% in the TE, which indicates clonal imbalances. Figure shows no statistically significant deviations between data and model. Panel F. Dependency of the statistical consistency on the cell arrest rate applied at the 4- and 8-cell stage. Harmonic mean P^{o} is maximal at an arrest rate of 0.3%. Panels G, H. P-P plots showing the predicted fraction of “1^{st} clone” cells in the TE and ICM against the observed fraction. Each data point represents one of the 66 blastocysts with statistical mean values μᵢ and SDs σᵢ as error bars. The data do not deviate significantly from a diagonal line. R² is the coefficient of determination

$${χ}_{red}^{2} = \frac{1}{66} {\sum }_{i = 1}^{66} {( \frac{{x}_{i} - {μ}_{i}}{{σ}_{i}} )}^{2}$$

is the reduced chi-squared test statistic, which is low in absence of statistical disagreement. Panel I. Overall degree of statistical consistency between data and model, measured by the harmonic mean of four P-values from B (whole embryo), C (ICM size), D (TE & ICM clonal distribution), as a function of the 2-cell clonal fate bias to become ICM. At about a 57:43 bias, the P^{o}=0.05 threshold is crossed. Therefore, an unbiased model was used throughout the figure. Overall, the model presented herein for human embryo development is also capable of reproducing early mouse embryogenesis within statistical errors and with appropriately adjusted numbers of ACD.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Retrospective lineage reconstruction of humans predicts that dramatic clonal imbalances in the body can be traced to the 2-cell stage embryo. However, whether and how such clonal asymmetries arise in the embryo are unclear. Disclosed herein are methods, compositions, and mathematical models for performing the first prospective lineage tracing of human embryos using live imaging, non-invasive cell labelling and computational predictions to determine the contribution of each 2-cell blastomere to the epiblast (body), hypoblast (yolk sac) and trophectoderm (placenta). The results demonstrate that most epiblast cells originate from only one 2-cell stage blastomere. This blastomere is the first to divide in the 2-cell stage human embryo and its descendants undertake most of the restricted number of epiblast-generating, asymmetric divisions at the 8-cell stage. The number of asymmetric cell divisions in early embryos is believed to be a bottleneck that determines the clonal composition of the human body. The methods, compositions, and mathematical models described herein provide insights for designing and building human embryo models. Some of the methods, compositions, and mathematical models disclosed herein are also described in “The first two blastomeres contribute unequally to the human embryo, Junyent, Sergi et al. Cell, Volume 187, Issue 11, 2838-2854.e17”, the content of which is hereby incorporated by reference in its entirety.

Disclosed herein include in vitro methods and composition for lineage tracing and imaging of live embryos as well as mathematical modeling methods for determining a clonal composition and cell distribution in blastocysts and generating in silico embryo models with clonal composition mimicking embryos. As described herein, the live embryos are live human embryos in some embodiments.

Disclosed herein includes a method for determining a clonal composition of a mammalian embryo such as a human embryo. In some embodiments, the method can comprise culturing a human embryo at the zygote stage in a first human embryo culture media until the human embryo forms 2-cell blastomeres, labeling one blastomere of the 2-cell blastomeres with a detectable lineage marker, culturing the 2-cell blastomeres in a second human embryo culture media for about 4 to 5 days allowing the 2-cell blastomeres to develop into a blastocyst, detecting cells expressing the detectable lineage marker in the blastocyst, and quantifying the clonal composition of the inner cell mass (ICM) and trophectoderm (TE) based on the detection of cells expressing the detectable lineage marker.

Disclosed herein also includes a method of selecting embryos. The method can comprise providing a plurality of human embryos at the zygote stage, determining a clonal composition of each human embryo of the plurality of human embryos according to the method disclosed herein, and selecting embryos having a desired clonal composition based on the percentage of cells expressing the detectable lineage marker and/or cells not expressing the detectable lineage marker in the inner cell mass and trophectoderm of an embryo at the blastocyst stage.

Disclosed herein also includes a computer-based method of determining a clonal composition in human embryo models. The method can comprise (i) generating a plurality of human embryo models each comprising two cells, wherein one cell of each human embryo model is randomly marked, (ii) modulating a set of parameters comprising a cell death rate, a cell arrest rate, and a number of asymmetric cell divisions for a stochastic model, (iii) subjecting the plurality of human embryo models to the stochastic model wherein each human embryo model undergoes successive rounds of cell division until the human embryo model reaches a desired total number of cells, and (iv) determining a clonal composition of the inner cell mass (ICM) and trophectoderm (TE) for each human embryo model reaching the desired total number of cells.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched. The term “enriched”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution.

As used herein the phrase “culture medium” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

### Mammalian Embryogenesis

Mammalian embryogenesis is the process of cell division and cellular differentiation during early prenatal development which leads to the development of a mammalian embryo. While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

So-called “Carnegie stages” have been established to describe stages of human development. Each stage is defined by the development of specific structures, and can be used to define equivalent stages in development of other species. The earliest Carnegie stages are as follows in Table 1:

TABLE 1
CARNEGIE STAGES OF DEVELOPMENT

| | Days since | |
|---|---|---|
| Carnegie | ovulation | |
| stage | (approx.) | Characteristic events/structures |
| 1 | 1 | fertilization; polar bodies |
| 2 | 2-3 | cleavage; morula; compaction |
| 3 | 4-5 | blastocyst and blastocoele; trophoblast |
| | | and embryoblast |
| 4 | 6 | syncytiotrophoblast; cytotrophoblast; |
| | | anchoring to endometrium |
| 5(a) | 7-8 | implantation; embryonic disc; bilaminar germ |
| | | disc; primary yolk sac |
| 5(b) | 9-10 | formation of trophoblast lacunae; complete |
| | | penetration into endometrium; amniotic |
| 5(c) | 11-16 | cavity; primary umbilical vesicle pre-chordal |
| | | plate; extra- embryonic mesoblast; |
| | | secondary yolk sac |
| 6 | 17 | primitive streak, primitive node, primitive |
| | | groove; secondary umbilical vesicle; |
| | | primordial germ cells; body stalk |

Human embryonic development is characterized by the processes of cell division and cellular differentiation of the embryo that occurs during the early stages of the development. A germinal stage of a human embryonic development refers to the time from fertilization through the development of the early embryo until implantation is completed in the uterus. The germinal stage takes about 10 days. During this stage, the one-celled zygote divides in a process referred to as cleavage. A blastocyst is then formed and implants in the uterus. Embryogenesis continues with the next stage of gastrulation, when the three germ layers of the embryo form in a process referred to as histogenesis, and the processes of neurulation and organogenesis follow.

In particular, following fertilization, the resulting one-celled zygote undergoes multiple mitotic cleavages, a series of mitotic divisions that occur after fertilization to create a multicellular embryo, resulting in the production of blastomeres (i.e., the dividing cells). The cleavage/cell division goes through a two-cell stage (approximately day one of cleavage), four-cell stage (approximately day two of cleavage), eight-cell stage (approximately day three of cleavage), and sixteen-cell stage (approximately day four of cleavage). The two-cell stage embryo comprises two blastomeres, the four-cell stage embryo comprises four blastomeres, the eight-cell stage embryo comprises eight blastomeres, and the sixteen-cell stage embryo comprises sixteen blastomeres, and so on. Initially, the dividing cells or blastomeres are undifferentiated and aggregated into a sphere enclosed within the zona pellucida of the embryo. When eight blastomeres have formed (8-cell stage), the cells start to compact and develop gap junctions, enabling them to develop in an integrated way and co-ordinate their response to physiological signals and environmental cues. A morula appears approximately four days after fertilization and refers to the solid sphere of cells within the zona pellucida when the cells number reaches sixteen (16-cell stage). Medically, this is often known as the final stage before the formation of a fluid-filled blastocoel cavity, which precedes blastula formation. Recent time-lapse microscopy observations suggest that compaction may represent an important checkpoint for human embryo viability, through which chromosomally abnormal blastomeres are sensed and eliminated by the embryo. Compaction is critical because it sets anatomical differences between cells (inner versus outer), ultimately determining their fate. The group of cells present in the center of the morula will eventually give rise to the inner cell mass and the embryo proper. The cells at the periphery, the outer cell mass cells, are critical in the cavitation of the morula that occurs as it transitions into a blastocyst.

Cleavage is the first stage in blastulation, the process of forming the blastocyst. A blastocyst refers to an embryo at the blastocyst stage. The term “blastocyst stage” as used herein refers to an early embryonic development stage that occurs around 5-6 days after fertilization and is characterized by a ball of cell containing about 50-150 cells and two distinct cell types of inner cell mass (ICM) and trophectoderm (TE) surrounded by a membrane called the zona pellucida. The ICM, also referred to as embryoblast, refers to a mass of cells inside the blastocyst that will eventually give rise to the definitive structures of the fetus. The ICM is surrounded by a single layer of trophoblast cells of the trophectoderm. The trophoblast cells form the outer layer of the blastocyst and line the inner side of the zona pellucida. Trophoblast cells are present four days after fertilization in humans and provide nutrients to the embryo and develop into a large part of the placenta.

The ICM and the TE will generate distinctly different cell types as implantation starts and embryogenesis continues. Trophectoderm cells form extraembryonic tissues, which act in a supporting role for the embryo proper. Furthermore, these cells pump fluid into the interior of the blastocyst, causing the formation of a polarized blastocyst with the ICM attached to the trophectoderm at one end. This polarization leaves a cavity, the blastocoel, creating the blastocyst structure. Accordingly, a blastocyst formation is characterized by the fluid-filled blastocoele, the ICM, and the fully differentiated trophectoderm-derived trophoblast. This difference in cellular localization causes the ICM cells exposed to the fluid cavity to adopt a primitive endoderm (or hypoblast) fate, while the remaining cells adopt a primitive ectoderm (or epiblast) fate. The hypoblast contributes to extraembryonic membranes and the epiblast will give rise to the ultimate embryo proper as well as some extraembryonic tissues. In some embodiments, the ICM can be used to predict the quality of an embryo during in vitro fertilization (IVF). The ICM's morphology is also a strong predictor of live birth after a frozen-thawed single embryo transfer.

### Lineage Tracing of Human Blastomeres

Provided herein include in vitro methods and compositions for lineage tracing of human blastomeres during various cell division stages (e.g., 2-cell stage, 4-cell stage, 8-cell stage, 16-cell stage, 32-cell stage, 64-cell stage, and beyond) and for determining their clonal composition of inner cell mass (ICM) and trophectoderm (TE). In some embodiments, the methods and compositions described herein can trace the cell division and clonal composition of ICM and TE in a human embryo from the zygote stage to the blastocyst stage. In some embodiments, the lineage tracing can be performed for a duration of 1-6 days (e.g., 1, 2, 3, 4, 5, or 6 days).

The method can comprise culturing a human embryo at the zygote stage (i.e., a human zygote) in a first human embryo culture media until the embryo forms a 2-cell stage embryo comprising two blastomeres, as a result of zygotic division. In some embodiments, the human zygote is a zygote having two pronucleic, also referred to a 2PN zygote. The human zygote can be cultured in the first human embryo culture media for about 12-20 hours until the completion of the first cleavage division.

The first human embryo culture media can comprise amino acids, physiological salts, energy substrates such as a carbon source, an antibiotic, and a buffer. In some embodiments, the carbon source comprises glucose. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. The human culture media can comprise a pH buffer such as biocarbonate or HEPES. Amino acids can comprise essential amino acids and non-essential amino acids. Exemplary essential amino acids can include valine, leucine, methionine, phenylalanine, tryptophan, threonine, histidine, and lysine. Exemplary non-essential amino acids can include L-glycine, L-alanine, L-asparagine, L-aspartic acid, L-glutamic acid, L-proline and L-serine. In some embodiments, the first human embryo culture media can comprise physiological salts, glucose, pH buffer (biocarbonate or HEPES), essential amino acids, non-essential amino acids, glutamine dipeptide, EDTA, gentamicin and water.

The first human embryo culture media may be free, substantially free, or essentially free of proteins. In some embodiments, the first human embryo culture media is not protein free and comprises a non-human serum or serum substitute. The non-human serum or serum substitute can comprise fetal bovine serum, bovine serum albumin, rat serum, KnockOut™ Serum Replacement, human serum albumin, or any combination thereof. In some embodiments, the first human embryo culture media can further comprise human α- and β-globulins. In some embodiments, the first human embryo culture media comprises a total protein concentration of about 10 mg/ml. In an exemplary embodiment, the first human embryo culture media can comprise human serum albumin, human α- and β-globulins, calcium chloride, sodium chloride, potassium chloride, potassium phosphate, magnesium sulfate, sodium bicarbonate, glucose, lactate Na salt, sodium pyruvate, amino acids, glycyl-glutamine, EDTA, gentamicin, water, or any combination thereof.

The method can further comprise labeling one blastomere of the 2-cell blastomeres with a detectable lineage marker. Any one of the two blastomeres can be randomly selected for labeling. The lineage marker is selected such that it does not affect the development of the embryo to the blastocyst stage and can enable annotation of the position and boundaries of cells in either the ICM or the TE. The blastomere of the embryo can be labeled, for example, by injecting the blastomere with an mRNA encoding the detectable lineage tracing marker. For example, the blastomere can be injected with an mRNA encoding a detectable lineage tracking marker that encodes a membrane targeting sequence. In an exemplary embodiment, one blastomere of each embryo, chosen at random, is injected with an mGap43-GFP mRNA using a Femtojet micro-injection system. After injection, mGAP43-GFP mRNA will be expressed in the labelled cell and all of that cell's descendants throughout preimplantation development.

The method can further comprise culturing the embryo, which comprises two blastomeres with one blastomere labeled and the other unlabeled, in a second human embryo culture media for about 4 to 5 days allowing the embryo to develop into a blastocyst. The blastocyst can be an expanded blastocyst or a non-expanded blastocyst. The term “expanded blastocyst” refers to a blastocyst with the inner cavity or blastocoel filled with fluid. Before the creation of the fluid space, the embryo is typically referred to as non-expanded. The first human embryo culture media and the second human embryo culture media can be the same or different.

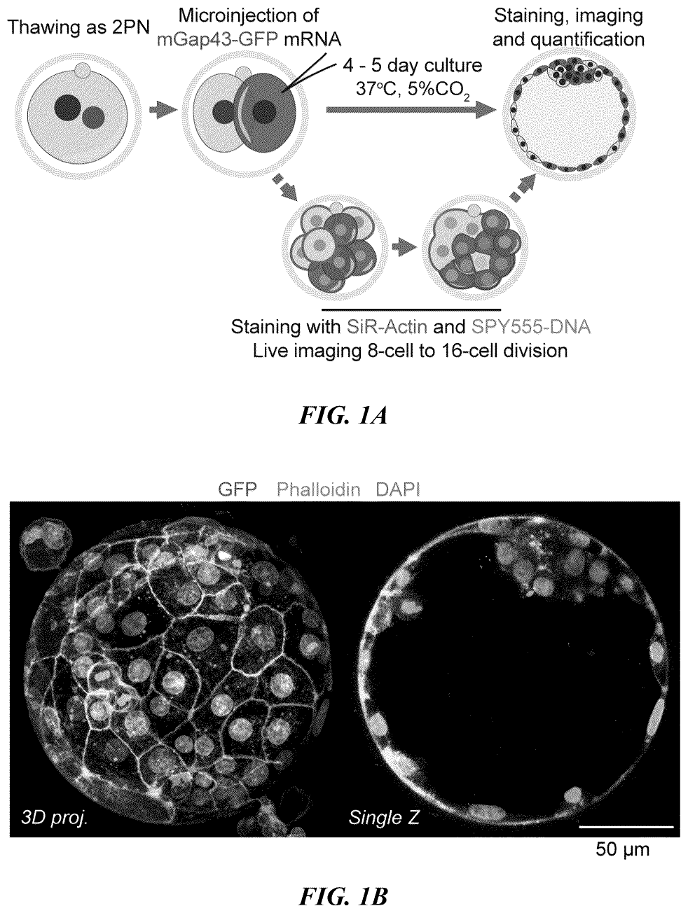

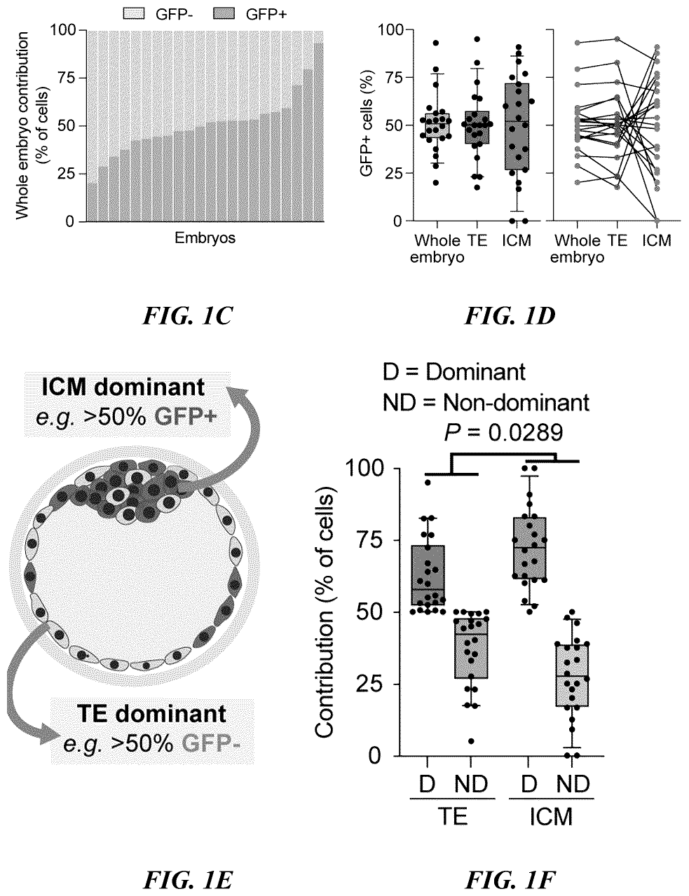

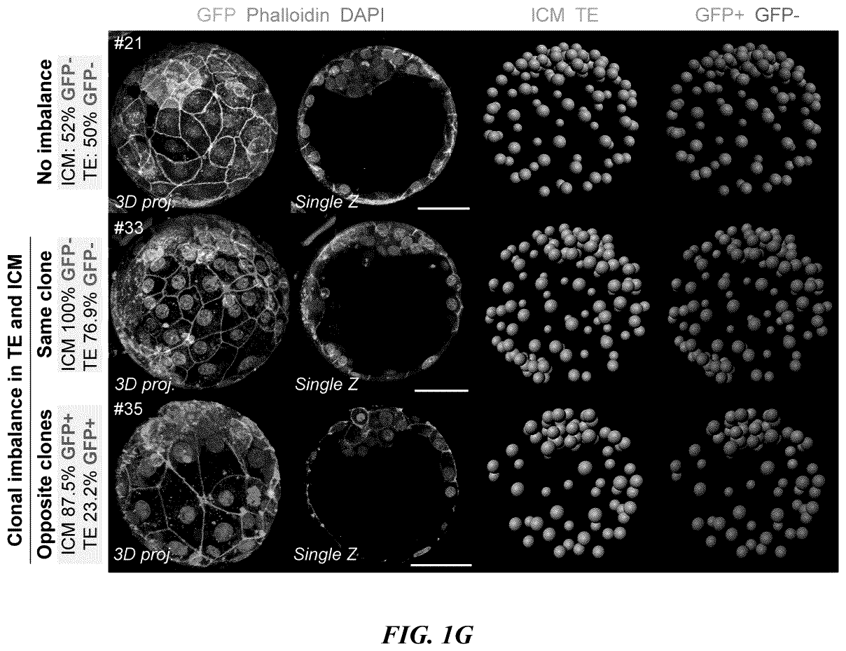

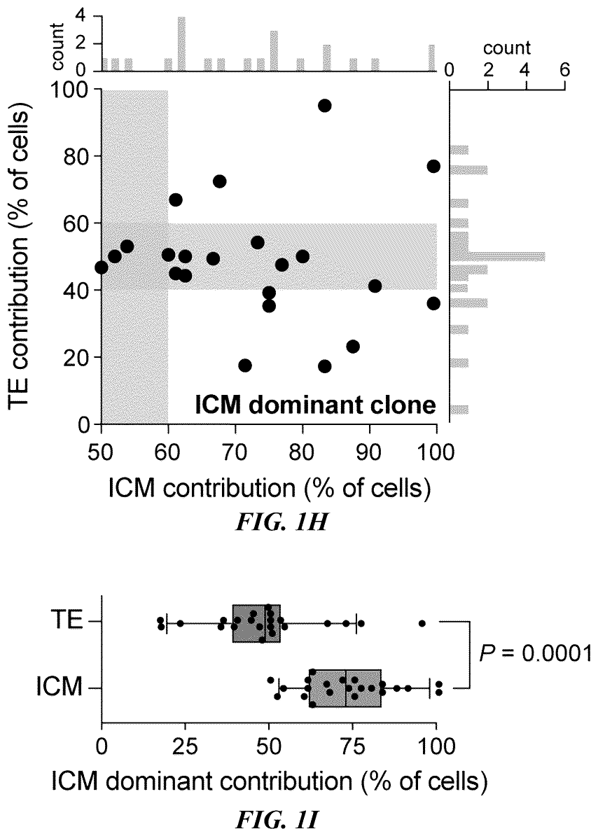

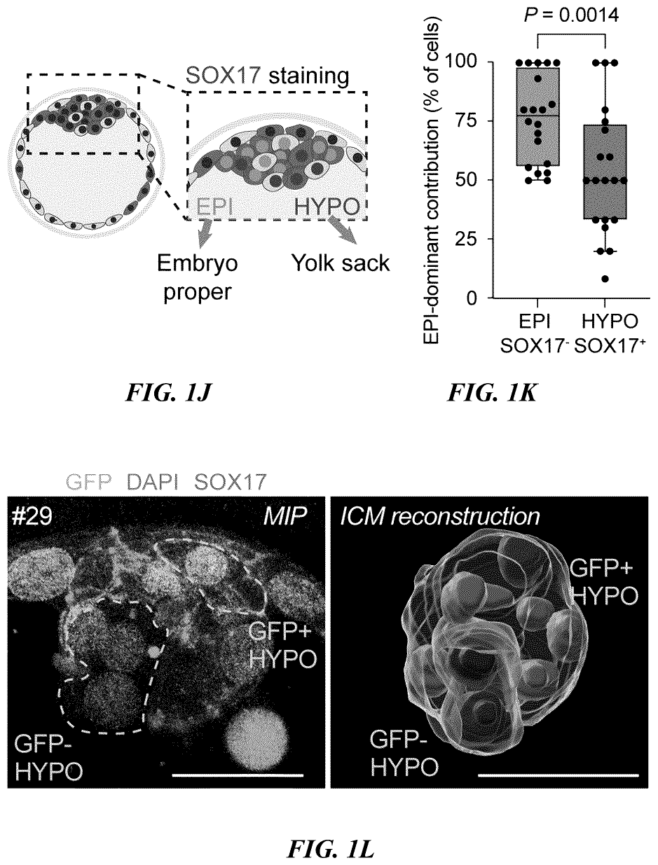

The human zygote can develop through a morula to a blastocyst stage during the embryo culturing. The human embryos will undergo successive rounds of cell division during the embryo culturing, forming 4-cell blastomeres (4-cell stage), 8-cell blastomeres (8-cell stage), 16-cell blastomeres (16-cell stage), 32-cell blastomeres (32-cell stage), 64-cell blastomeres (64-cell stage) and so on, until the human embryos reach the blastocyst stage or beyond. In some embodiments, a subset of embryos at the 2-cell stage, 4-cell stage, 8-cell stage, and/or 16-cell stage (i.e., 2-cell blastomeres, 4-cell blastomeres, 8-cell blastomeres, and 16-cell blastomeres) can be selected from the culture media prior to the formation of a blastocyst and live-stained and imaged (FIG. 1A). Staining the embryos can comprise live staining the embryos in a human embryo culture media containing dyes such as membrane-permeable fluorescent dyes. The human embryo culture media can be the same as the second human embryo culture media and/or the first human embryo culture media. Suitable dyes can be selected to track both genomic nucleic acids and components of cytoskeleton (e.g., F-actin) to enable co-labeling of nuclear and membrane. In some embodiments, the embryos can be transferred to the same medium containing the dyes at a different concentration such as at a lower concentration. The embryos can be stained for about 25-28 hours post-injection and imaged for a desired period of time, for example, until the embryos form blastocysts. The position and division of each cell in the embryo can be monitored and tracked over time. In an exemplary embodiment, an embryo at the 8-cell stage can be stained in a human embryo culture media containing SiR-Actin and SPY555-DNA at 27 h post-injection and imaged them for a further 28 h.

In some embodiments, the method can further comprise monitoring asymmetric cell division and/or symmetric cell division events during the cell stage transition (e.g., 2- to 4-cell transition, 4- to 8-cell transition, 8- to 16-cell transition, etc.). An asymmetric cell division (ACD) is defined as a cell division leading to the ingression of one daughter cell to allocate an ICM cell. A symmetric cell division (SCD) is defined as a cell division wherein both daughter cells remain at the embryo surface). Monitoring ACD and/or SCD events can comprise counting the number of ACD events or the number of cell internalizations. ACD and SCD can be verified, for example, by measuring the angle of division of the cells. Exemplary embodiments on ACD observation and quantification can be found, for example, in Example 4. The exemplary data indicate that the clonal composition of the ACD strongly predicts the clonal composition of the ICM at the blastocyst stage. Accordingly, early cell ingression to the ICM is considered as a strong predictor of ICM clonal composition.

The method described herein can further comprise imaging the embryos by performing time-lapse imaging analysis. In some embodiments, the embryos can be cultured in micro-well culture dishes, in which each micro-well holds a single embryo cell, and the bottom surface of each micro-well has an optical quality finish such that the entire group of embryos within a single dish can be imaged simultaneously by a single miniature microscope within sufficient solution to follow the cell mitosis processes. Images are acquired over time, and then analyzed to determine measurements of parameters such as cell numbers, size, positions, ACD events, and/or other parameters of interest and/or described herein. Time-lapse imaging can be performed with any computer-controlled microscope that is equipped for digital image storage and analysis as will be understood by a person skilled in the art.

The method can further comprise identifying cells expressing the detectable lineage marker in each embryo (e.g., blastocyst). The activity or level of a lineage marker protein can be detected and/or quantified by detecting or quantifying the expressed marker polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-marker protein antibodies, such as intrabodies, may also be used for imaging purposes, for example, to detect the presence of marker protein in cells or, e.g., an EP structure. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a K_{d} of at most about 10⁻⁶M, 10⁻⁷M, 10⁻⁸M, 10⁻⁹ M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or can be prepared by methods known in the art. A list of antibodies that can be used to assay the presence, absence, level, and localization of one or more of the linage markers described herein are listed in Table 2.

Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

The method can further comprise quantifying the clonal composition of the inner cell mass (ICM) and trophectoderm (TE) in the blastocyst. The clonal composition of the TE and ICM (including epiblast and hydroblast) can be quantified according to the contribution of either cell population, marked or unmarked, to each compartment (ICM or TE). The marked cell population comprises cells expressing a detectable lineage marker and is derived from the parent blastomere marked with the detectable lineage marker, while the unmarked cell population comprises cells not expressing the detectable lineage marker and is derived from the unmarked blastomere.

As used herein, a clone typically refers to a group of genetically identical cells that are derived from a single cell such as a single blastomere at the 2-cell stage. The term “clonal composition” refers to the relative abundance or distribution of different cell populations within a group of cells such as cells of a whole embryo or cells of individual compartments in the whole embryo (e.g., ICM or TE). In the instant case, the clonal composition can be used to track the evolution of each blastomere from the 2-cell blastomere stage till the blastocyst formation and to determine how each blastomere contributes to the cells within ICM or TE in the blastocyst stage. In some embodiments, the clonal composition determination can be achieved by counting the number of cells derived from each blastomere of the 2-cell stage in different compartments of an embryo such as in the ICM and TE of the embryo. The most abundant clone (i.e., greater than 50% cells) in the ICM and TE is labeled as “ICM dominant” and “TE dominant”, respectively. The marked and unmarked cells (e.g., GFP+ and GFP− cells) in the ICM and TE can be counted, for example, by an assessor. In some embodiments, only the presence or absence of the lineage marker in a cell is assessed, rather than the marker intensity. The marked and unmarked cell counts are used to quantify the clonal composition of the TE and ICM (including epiblast and hydroblast). In some embodiments, the clonal composition quantification can further comprise identifying the dominant population (i.e., marked or unmarked) in the ICM and/or TE of each blastocyst, thereby identifying which one of the two-cell blastomeres contributes dominantly to the ICM and/or the TE compartment of a blastocyst. The dominant population in the ICM and/or TE of a blastocyst can be identified by determining the percentage of marked cells and/or unmarked cells in the ICM and/or TE. Accordingly, in some embodiments, the method comprises determining the percentage of marked cells and/or unmarked cells in the ICM and/or TE of the blastocyst. The dominant population in the ICM can be compared to the contribution of the dominant population in either the polar or the mural TE. Accordingly, in some embodiments, the method can further comprise identifying the number, size, and/or position of a TE cell or cell cluster such as polar TE and/or mural TE.

In some embodiments, the two blastomeres at the 2-cell stage do not contribute equally to the cells in the ICM and TE (e.g., polar TE) of a blastocyst, resulting in clonal imbalance or clonal asymmetry. In some embodiments, greater than 50% (e.g., 51%, 55%, 60%, 65%, 70%, 75% or greater) of the cells in the ICM of the blastocyst are derived from one blastomere of the two blastomeres. In some embodiments, both blastomeres contribute equally or substantially equally (about 50%) to the ICM, also referred to clonal symmetry.

The blastocysts can be given a score using an embryo grading system, such as the Gardner blastocyst grading system. An embryo grading system typically assesses the quality of the embryos based on factors related to the inner cell mass, the trophectoderm, the blastocoel cavity, the zona pellucida, and how tight cells are packed. The Gardner blastocyst grading system can assign three separate quality scores to each blastocyst embryo for (1) blastocyst development stage status, including the size of the blastocoel cavity, the expansion and hatching status; (2) inner cell mass score or quality, such as the number of cells and how the cells are packed; and (3) trophectoderm score or quality, including the number of cells and whether a cohesive layer is formed. In some embodiments, the method can further comprise selecting blastocysts with a desired quality score.

The method can further comprise performing blastocyst ploidy analysis. Blastocysts (e.g., expanded blastocysts) can be separating into an embryonic part containing the inner mass and an abembryonic part containing mural TE. Pre-implantation Genetic Testing for Aneuploidy (PGT-A) analysis can then be performed as will be understood by a person skilled in the art.

In some embodiments, a method of selecting embryos based on their clonal composition is described. The method can comprise providing a plurality of human embryos at the zygote stage and determining the clonal composition of each human embryo according to the methods described herein. Embryos with a desired clonal composition can be selected based on the percentage of cells expressing the detectable lineage marker and cells not expressing the detectable lineage marker in the ICM and/or TE compartment of the embryo at its blastocyst stage. In some embodiments, the selected embryo comprises clonally imbalanced inner cell mass. In some other embodiments, the ICM compartment of the selected embryos is clonally symmetric, i.e., cells in the ICM compartment are derived equally or substantially equally from the two blastomeres at the 2-cell stage.

In some embodiments, a method for investigating the effect of a test agent on embryonic development is described. The method can comprise contacting a test agent with a human embryo at the zygote stage, determining a clonal composition of the human embryo according to the method described herein, and determining the effect of the test agent on the clonal composition. The determining step can comprise comparing the clonal composition obtained in the presence of the test agent with a clonal composition obtained in the absence of the test agent.

### A Statistical Model of Blastocyst Development

Provided herein also includes a statistic model and related methods of determining clonal symmetry or clonal composition in mammalian embryos (e.g., human embryos). The model and related methods described herein can bypass sample size limits through computer simulations and predict cell distributions and clonal asymmetry using a set of parameters including, for example, a cell death rate, a cell arrest rate, and a number of asymmetric cell divisions (i.e., the number of cells internalizing to the inner cell mass of an embryo during each cell division). The parameters can be adjusted following estimates available in the literature and/or the in vitro data obtained using the methods described in the previous section.

In some embodiments, a stochastic model (e.g., Markov Chain Monte Carlo model) of blastocyst development is developed to determine the distributions of marked and unmarked cells in the whole embryo and in the inner cell mass and/or the trophectoderm. As will be understood by a person skilled in the art, a stochastic model is a method for predicting statistical properties of possible outcomes by accounting for random variance in one or more parameters over time. Examples of stochastic modeling methods include, for example, Monte Carlo simulations, Markov chains, Poisson processes, random walks, Brownian motion, Regression models, and others identifiable to a person skilled in the art.

A pool of embryos can be generated with each embryo at the two-cell stage, with one blastomere marked and the other unmarked. The embryos can then undergo consecutive rounds of cell division during which the cell type (marked or unmarked) is inherited by the daughter cells using the stochastic model. At the 4-cell and 8-cell stages, a random subset of the blastomeres are let arrest (i.e., not divide any further), and from the 64-cell stage onward another random subset of blastomeres are let die. The subset of blastomeres that are let die may or may not include the already arrested cells. Both cell selection processes for arrest and death are assumed to be independent Bernoulli processes with a predefined cell arrest probability and a predefined cell death probability in each affected cleavage cycle. Thus, the fraction of blastomeres undergoing arrest or death is not fixed at the predefined cell arrest probability and cell death probability, but varies across these probabilities according to a binomial distribution. Cell arrest and death are assumed to occur to marked and unmarked blastomeres, as well as to those in the ICM and TE, with equal chance.

During the 8- to 16-, 16- to 32- and 32- to 64-cell stage transitions, a subset of the daughter blastomeres is randomly selected and internalized, generating a potential lineage imbalance. From this stage onward, the ICM and TE cell pools continue dividing separately. The number of cells internalizing (i.e., the number of asymmetric cell divisions) is not fixed, but drawn uniformly within a predefined range, [nₘᵢₙ, nₘₐₓ], in each cleavage generation. The value can be selected based on observations in in vitro human embryos, and is typically in the range between 0 and 3. In some embodiments, the nₘᵢₙ can be set to 1, 0, 0 for the 8- to 16-, 16- to 32- and 32- to 64-cell stage transition, respectively. The nₘₐₓ can be set to 3, 2, and 1 for the 8- to 16-, 16- to 32- and 32- to 64-cell stage transition, respectively. In some embodiments, the number of asymmetric cell divisions selected herein can reproduce the observed fraction of ICM cells of the total cells in in vitro embryos.

In some embodiments, a computer-based method of determining a clonal composition in human embryo models is described. The method can comprise (i) generating a plurality of human embryo models each comprising two cells, wherein one cell of each human embryo model is randomly marked, (ii) modulating a set of parameters comprising a cell death rate, a cell arrest rate, and a number of asymmetric cell divisions for a stochastic model, (iii) subjecting the plurality of human embryo models to the stochastic model wherein each human embryo model undergoes successive rounds of cell division until the human embryo model reaches a desired total number of cells, and (iv) determining a clonal composition of the inner cell mass (ICM) and trophectoderm (TE) for each human embryo model.

In some embodiments, the plurality of human embryo models can undergo 2, 3, 4, 5, 6, 7, 8, 9, 10, or more rounds of cell divisions until the embryos reach a desired final number of blastomeres, such as the number of blastomeres observed in a human dataset. In some embodiments, the plurality of human embryo models can undergo at least 5 rounds of cell divisions, i.e., from the 2-cell stage to the 4-cell stage, from the 4-cell stage to the 8-cell stage, from the 8-cell stage to the 16-cell stage, from the 16-cell stage to the 32-cell stage, from the 32-cell stage to the 64-cell stage. In some embodiments, the desired final number of blastomeres is at least 64 or greater.

In some embodiments, the steps described in the computer-based method (i.e., steps (i)-(iv)) can be repeated for at least 2, 5, 10, 100, 1000, 10⁴, 10⁵, 10⁶, 10⁷, 10⁸, 10⁹, or a number or range between any two of these value, times. Repeating this process can allow one to evaluate the statistically expected distribution of marked and unmarked cells in the whole embryos as well as in the ICM and TE individually.

The clonal composition of the whole embryos and in individual ICM and TE can be evaluated at early stages, for example, at the 16-cell stage after the first asymmetric cell division event. The clonal composition at early stages can be compared to the final outcome at the blastocyst stage to study how the early ACD affects the clonal dominance in the ICM at the blastocyst stage in the models.

The values of the cell death rate, the cell arrest rate, and/or the number of asymmetric cell divisions (ACDs) can be predefined, for example, based on literature curation and/or the in vitro data. For example, the literature indicates that death randomly affects any embryo cell from the 64-cell stage onwards, at an average rate of about 4.4%. Accordingly, in some embodiments, the cell death rate can be predefined as about 4.4%, resulting in an average of about 7-8% dead cells per blastocyst. Accordingly, in some embodiments, modulating the set of parameters comprises selecting the cell death rate such that the average percentage of dead cells at the blastocyst state is in the range of 7-8%. The cell death rate can be selected for cell divisions beyond the 64-cell stage.

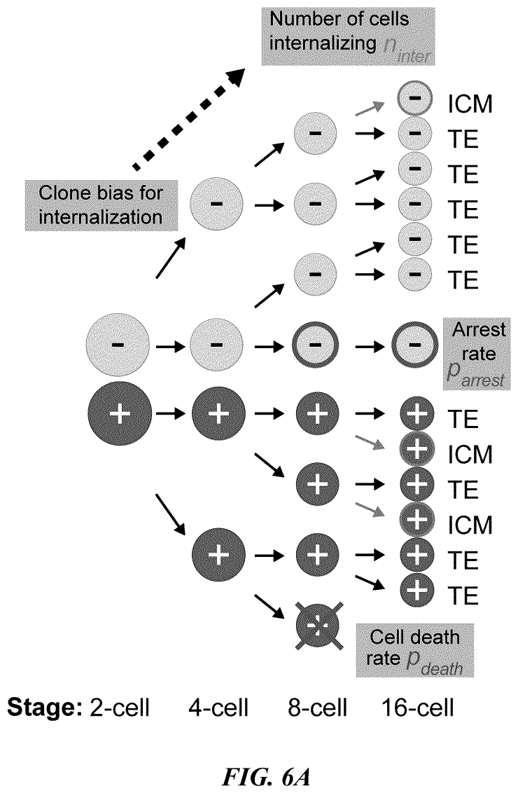

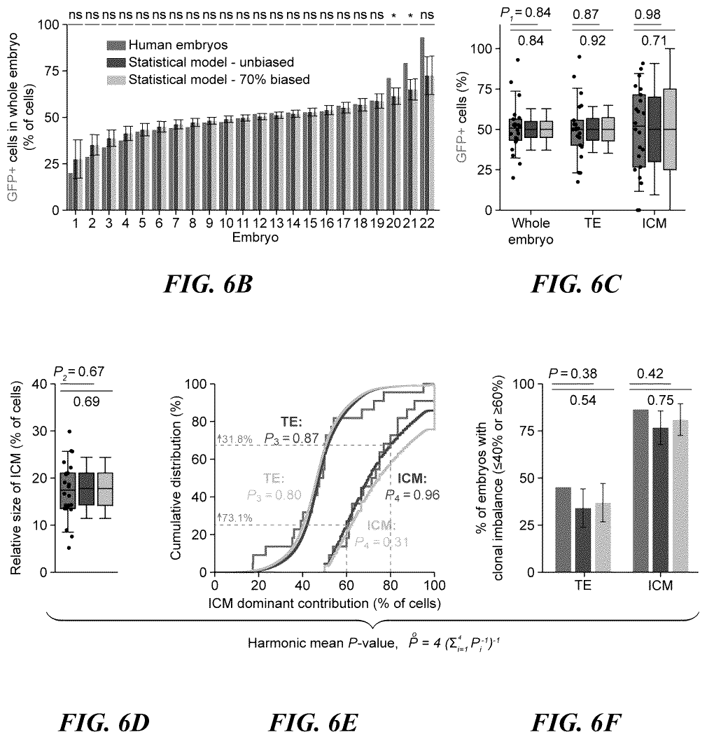

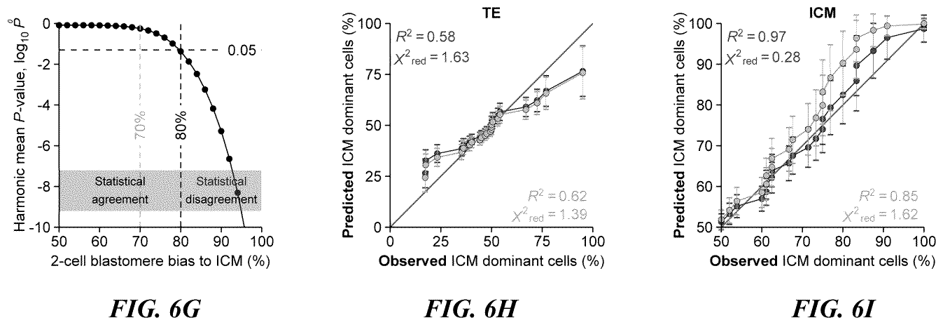

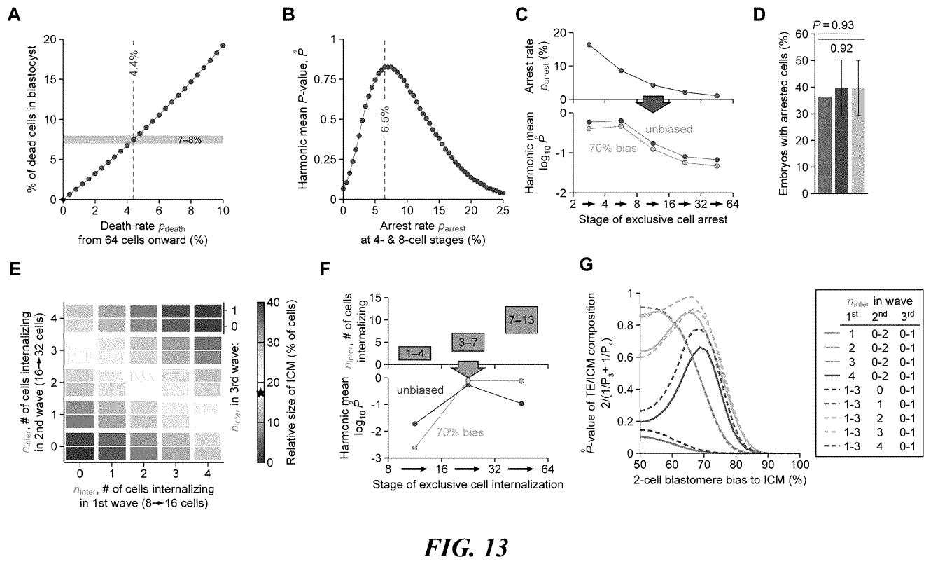

The cell arrest rate can be selected based on a maximization of the statistical agreement between the model prediction and the in vitro data (see, for example, FIG. 13, panel B), as quantified by the harmonic mean P-value from four two-sample Kolmogorov-Smirnov tests that compare the clonal distributions across the whole embryos, the distributions of relative ICM sizes, and the distributions of the shares of the ICM dominant clone in the TE and ICM (see, for example, FIGS. 6C-E). In some embodiments, a cell arrest rate is selected as a value of about 6.5% affecting cells at the 4- or 8-cell stage. Accordingly, in some embodiments, modulating the set of parameters comprises selecting the cell arrest rate at the 4-cell stage and/or the 8-cell stage. In some embodiments, the cell arrest rate is set to a value of about 6.5%.

The number of asymmetric cell divisions (ACDs) or the number of cells internalizing in each division generation from 8- to 64-cell stages can be individually specified for the 8- to 16-cell transition, 16- to 32-cell transition, and/or 32- to 64-cell transition. For example, the number of asymmetric cell divisions is specified as 0, 1, 2, or 3. In some embodiments, the number of asymmetric cell divisions for the 8- to 16-cell transition is set as 1, 2 or 3, the number of asymmetric cell divisions for the 16- to 32-cell transition is set as 1 or 2, and/or the number of asymmetric cell divisions for the 32- to 64-cell transition is set as 0 or 1. In some embodiments, the number of ACDs for the 8- to 16-cell transition, the 16- to 32-cell transition, and the 32- to 64-cell transition is set to 1, 0, and 0, respectively. In some embodiments, the number of ACDs for the 8- to 16-cell transition, the 16- to 32-cell transition, and the 32- to 64-cell transition is set to 3, 2, and 1 respectively. In some embodiments, the number of asymmetric cell division prior to the 8-cell stage is set to zero. The number of asymmetric cell division after the 64-cell stage can also be set to zero.

In some embodiments, the marked and unmarked cells can be randomly picked for internalization during these cleavage cycles, thus referred to as “unbiased” lineage determination. In some embodiments, one cell type of the two cell types (marked or unmarked) is selected to have a higher probability for ACD than the other cell type. Blastomeres are still randomly selected for internalization at the same total number (e.g., 1-3), but with unequal probability for marked and unmarked clones. Accordingly, in some embodiments, the set of parameters further comprises a fate determination bias which controls the average ratio between internalization of marked and unmarked cells. In some embodiments, the fate determination bias can be selected as having a value between 0.5-0.8. A fate determination bias with a value of 0.5 indicates an unbiased selection, i.e., equal average likelihood for marked and unmarked blastomeres to internalize, while a fate determination bias with a value of 1 represents exclusive internalization of one of the two cell types.

Accordingly, in some embodiments, the two cell types of a human embryo model has equal or substantially equal probability for an asymmetric cell division event. In some other embodiments, the two cell types of a human embryo model has unequal probability for an asymmetric cell division.

In some embodiments, subjecting the plurality of human embryo models to the stochastic model comprises randomly selecting a subset of cells for cell death based on the selected cell death rate for cell divisions from the 64-cell stage and beyond. In some embodiments, subjecting the plurality of human embryo models to the stochastic model comprises randomly selecting a subset of cells for cell arrest based on the selected cell arrest rate at the 4-cell or 8-cell stage. The selected subset of cells for cell death may or may not overlap with the selected subset of cells for cell arrest.

In some embodiments, modulating the set of parameters comprises fitting the set of parameters to in vitro clonal composition data. The in vitro clonal composition data can be obtained using the in vitro method described herein in the previous section. For example, to fit the set of parameters to the in vitro clonal composition data, a predefined value is selected for one or more parameters (e.g., the cell death rate, the cell arrest rate, and/or the number of ACDs). Next, the plurality of human embryo models can be subjected to the stochastic model until the human embryo model reaches a desired total number of cells. The clonal composition of the ICM and TE can be determined for the human embryo model, and then compared to the in vitro clonal composition data. The preceding steps can be repeated until the clonal composition determined by the model matches with the in vitro clonal composition.

The statistical models and related methods presented herein for human embryo development are capable of reproducing early mammalian embryogenesis within statistical errors and with appropriately adjusted numbers of ACD.

The statistical models and related methods presented herein can also be used to study embryos from other suitable mammalian species, such as: primates, including humans, great apes (e.g., gorillas, chimpanzees, orangutans), old world monkeys, new world monkeys; rodents (e.g., mice, rats, guinea pigs, hamsters); cats; dogs; lagomorphs (including rabbits); cows; sheep; goats; horses; pigs; and any other livestock, agricultural, laboratory or domestic mammals. The presently disclosed compositions, methods, and computational models may be used to produce blastocysts and blastocyst models from any human or non-human mammal, including but not limited to those described above.

### EXAMPLES

Some aspects of the embodiments discussed above are disclosed in further detail in the following examples, which are not in any way intended to limit the scope of the present disclosure.

### Example 1

### General Experimental Models and Methods

This example describes experimental models, general methods, and related quantification and statistical analysis used in Examples 2-7 below.

### Mouse Embryo Recovery and Culture.

Ethical approval for studies on mouse embryos was received from the Institutional Animal Care and Use Committee (IACUC) at the California Institute of Technology. To obtain mouse embryos at the 2-cell stage, 4-week-old B6S females (B6SJLF1/J) were stimulated to superovulate by intraperitoneal injection of pregnant mare serum gonadotrophin (PMSG; 10IU; Prospec, HOR-272) and, 48 h later, intraperitoneal injection of human chorionic gonadotrophin (hCG; 10IU; Chorulon, 031345). Immediately after hCG injection, females were mated with B6S males. The females were sacrificed 45 hours post hCG to obtain 2-cell embryos. Oviducts were isolated in M2 medium (Millipore-Sigma, M7167-100ML) and cut into pieces with fine scissors. For longer culture, embryos were transferred to drops of preequilibrated EmbryoMax® Advanced KSOM Medium (Sigma, MR-101-D) and cultured in standard conditions (37° C., 5% CO₂) under mineral oil (Irvine Scientific, 9305).

### Human Embryo Recovery and Culture.

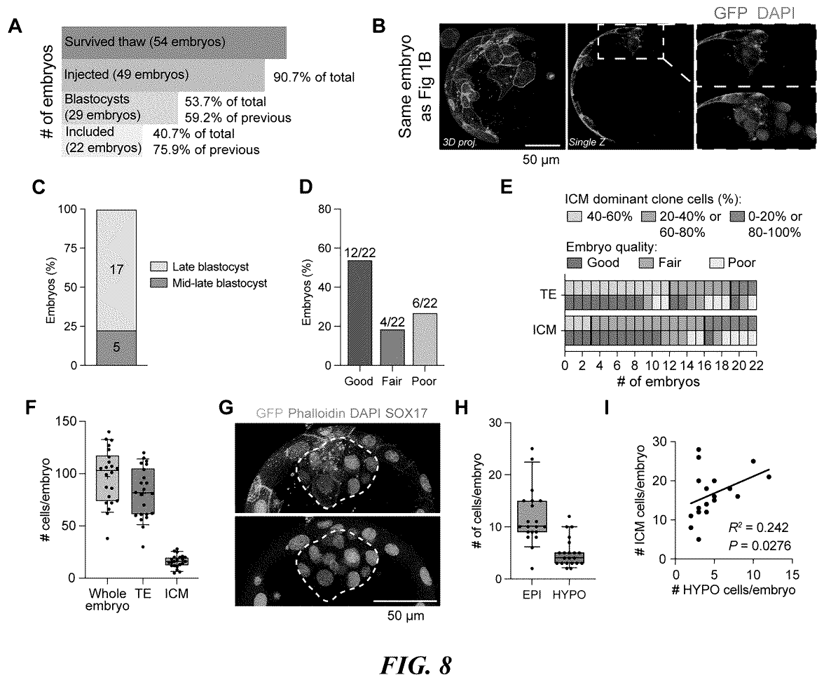

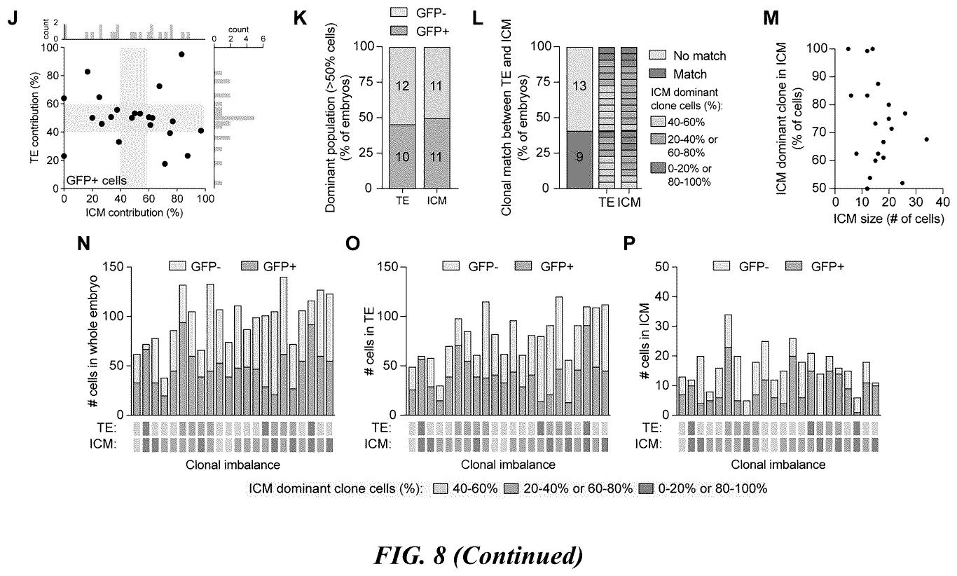

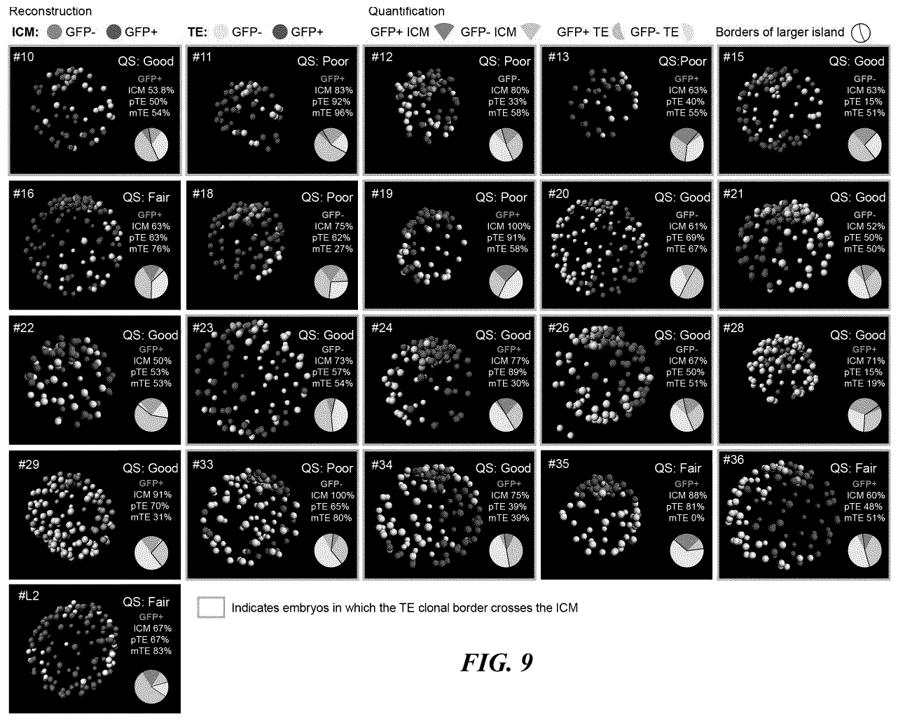

Human embryo work was approved by the California Institute of Technology Committee for the Protection of Human Subjects (IRB protocol numbers 19-0948 and 22-0101, approved before the experiments were performed). Human embryos at the zygote stage were obtained from HRC Fertility. Supernumerary cryopreserved embryos were donated after completion of IVF. They were not created for research purposes. Patients were informed of the conditions of the donation, objectives, and methodology of human embryo research. They were offered counselling and alternative options, including discarding embryos and continued cryopreservation. Patients were informed that they would not benefit directly from the donation of embryos to research. A total of 54 anonymized donated human embryos at the zygote two pronuclei stage (day 1 post-fertilization) were used. Embryos were thawed using Quinn's Advantage™ Embryo Thaw Kit (CooperSurgical, ART-8016) as per the manufacturer's instructions. Subsequently, the embryos were transferred to preequilibrated (overnight, 37° C., 5% CO₂) Global® human embryo culture media (CooperSurgical, LGGG-050) and cultured in standard culture conditions (37° C., 5% CO₂) under mineral oil (Irvine Scientific, 9305) for 12-20 h (˜17 h), until they reached 2-cell stage. Five embryos degenerated before the completion of the first zygotic division and could not be used for the study. Of the remaining 49 embryos, 29 developed to the blastocyst stage (FIG. 8, panel A). This ratio of successful in vitro human embryo culture is in line with previous reports.

The dataset obtained is unique, as access to human zygotes is extremely limited for several reasons: (1) research-consented embryos can only be sourced from IVF clinics and their availability relies on patient donation; (2) the practice of zygote freezing is no longer commonly used and the current practice is to culture embryos to the blastocyst stage, at which point assessments of embryo quality can be performed, and then frozen for subsequent IVF cycles. Thus, the pool of zygote stage embryos that is available for research is extremely limited and may be even smaller in the future. Embryos at other stages are unusable for the purpose of this study, as clonal origin cannot be identified for lineage prospective studies.

### Embryoscope™ Time-Lapse Movies

Embryoscope™ time-lapse movies of human embryos from the zygote to blastocyst stage were provided by IVIRMA-Valencia (IVI Foundation, Spain). These movies were generated during routine IVF clinical practice, when the embryos were placed in time-lapse incubators. The use of movies for retrospective analysis was approved by the Research Ethics Committee of IVI Valencia (IRB protocol number 2203-VLC-028-MD). Embryo imaging preceded the work presented in this disclosure, and this project had no impact on the culture practices of the embryos analyzed. The movies were received by the researchers anonymized. The IRB approval of the protocol 2203-VLC-028-MD stated that informed consent letters were “not applicable, as this is a retrospective study that carried out exclusively with anonymized or pseudonymized data”. IRB approval ensured that the proposed project fulfilled the requirements of Organic Law 3/2018, of 5 December, on Personal Data Protection and guarantee of digital rights (Spain), Regulation (EU) 2016/679 of the European Parliament and of the Council of 27 Apr. 2016 on the protection of individuals with regard to the processing of personal data and on the free movement of such data, Law 14/2007, of 3 July, on Biomedical Research (Spain), Law 14/2006, of 26 May, on Assisted Reproduction Techniques (Spain), as well as the regulations that develop them. The California Institute of Technology Committee for the Protection of Human Subjects assessment of the use of these data in the retrospective analysis (application 21-1177) confirmed that the work was exempt of IRB approval as it represented secondary research for which consent is not required, as specified in the U.S. Federal Code (45 C.F.R. § 46.104).

Microinjection of mGap43-GFP mRNA for Cell Labelling

mRNA encoding a membrane targeting sequence of GAP43 fused with GFP (mGap43-GFP) was prepared by in vitro transcription with the mMessage mMachine T3 kit (Thermo Fischer Scientific, AM1348) from linearized pRN3p plasmid containing mGap43-GFP (Plasmid #139402, Addgene) sequence as previously described. Human embryos at the 2-cell stage were transferred to Multipurpose Handling Medium-Complete (MHM-C) with Gentamicin (Irvine Scientific, 90166) for microinjection. One blastomere of each embryo, chosen at random, was injected with a minimal volume of 100 ng/μL of mGap43-GFP mRNA using a Femtojet micro-injection system (Eppendorf) with negative capacitance. It has been previously shown that this method does not affect the developmental potential of mouse or human blastomeres. The technique of microinjection of mGap43-GFP mRNA for use as a lineage tracing marker, in both mouse and human embryos, has been well reported. The previously optimized concentration of mGAP43-GFP in human embryos was verified in a pilot experiment, ensuring that it would result in a clear membrane signal in all cells developing from the injected blastomere and that it would not affect the development of the embryo to the blastocyst stage. A membrane marker would enable annotation of the position and boundaries of cells in either the ICM or the TE. The embryo microinjection in all the experiments was performed by an expert, with over a decade of experience in micromanipulation of mammalian embryos. Furthermore, microinjection of human embryos had successfully been performed using the same system previously. Following microinjection, embryos were transferred to fresh drops of preequilibrated (37° C., 5% CO₂) Global® human embryo culture media under mineral oil for culture for 4 (to Day 5 of development) or 5 (to Day 6 of development) days, until the formation of an expanded blastocyst (at 37° C., 5% CO₂).

### Fixation and Staining

Embryos were fixed in freshly prepared 4% PFA in PBS for 20 min at room temperature (RT). Fixed embryos were washed twice in PBS without Mg²⁺/Ca²⁺ (PBS) containing 0.1% Tween-20 (PBST). Embryos were permeabilized in PBS containing 0.3% Triton X-100 and 0.1M Glycine for 20 min (RT) and incubated overnight (4° C.) in PBST with Alexa Fluor® 647 Phalloidin (1:400, ThermoFisher Scientific, A12381) and DAPI (ThermoFisher Scientific, D3571) to visualize the F-actin cytoskeleton and nuclei, respectively. Subsequently, they were washed 3 times in PBST and 2 times in M2 medium and transferred to drops of M2 medium under mineral oil on glass-bottom dishes for imaging. Images were taken on a Leica SP8 confocal, using a 40× water immersion objective (N. A.=1.10).

For immunostaining, embryos were washed 3 times in PBST and incubated in blocking buffer (PBST containing 1% fetal calf serum) for 1 h (RT). Embryos were moved to blocking buffer containing Goat anti-SOX17 primary antibody (1:250, R&D systems, AF1924) overnight at 4° C. and then washed in PBST (3×15 min) and incubated with secondary antibody (Donkey anti-Goat AF568, 1:1,000 in blocking buffer, ThermoFisher Scientific, A-11057) at RT for 2 h. Subsequently, embryos were washed in PBST (3×5 min) and in drops of M2 medium (2×5 min) and moved to drops of M2 medium under mineral oil on glass-bottom dishes for imaging, as described above. Two embryos were unavailable for SOX17 staining and were excluded from the relevant figures.

Live Staining with SiR-Actin and SPY555-DNA Dyes

For live staining, human embryos were placed in 10-20 μL drops of preequilibrated Global® human embryo culture media containing SiR-Actin (1 μM; Cytoskeleton Inc., CY-SC001) and SPY555-DNA (2×; Cytoskeleton Inc., CY-SC201) dyes. After 2 h of incubation, embryos were transferred to small drops of the same medium containing both dyes at a lower concentration (100 nM SiR-Actin and 0.2×SPY555-DNA), under mineral oil, on u-Dish 35 mm, high Glass Bottom dishes (Ibidi, 81158) for time-lapse live imaging.

To stain mouse embryos, the same steps were followed but the media used was EmbryoMax® Advanced KSOM, preequilibrated at 37° C., 5% CO₂. Recent publications report the use of the same dyes in human embryos, at equivalent concentrations as the ones used in this study, without deleterious effects.

### Time-Lapse Live Imaging

For time-lapse live imaging, a Zeiss LSM980 confocal microscope equipped with a culture chamber module stabilized at 37° C., 5% CO₂ was used. Embryos were imaged with the following parameters: frequency: 30 min; 18 planes per stack; z-step: 5 μm; objective: 40×, objective N.A.=1.2.

### Human Blastocyst Ploidy Analysis

Zona pellucida were removed from blastocysts by treatment with Acidic Tyrode's Solution (Sigma, T1788), followed by three washes in M2 medium. The blastocysts were transferred to fresh drops of M2 medium and split with a tungsten wire needle into the embryonic part (ICM+polar TE) and abembryonic part (mural TE). Each resulting tissue/sample was washed separately through a fresh drop of ultrapure MiliQ water and collected in a PCR tube containing 5 μL H₂O. Subsequently, 1 μL of lysis buffer (200 mM KOH, 50 mM DTT in MiliQ H2O) was added to each PCR tube. The samples were incubated at 65° C. for 10 min. Following this, the samples were spun down and 1 μL of neutralization buffer (0.9 M Tris base pH 8.3, 0.3 M KCl, 0.2 M HCl in MiliQ H₂O) was added to each tube. The samples were stored in −20° C. prior to the Pre-implantation Genetic Testing for Aneuploidy (PGT-A) analysis.

PGT-A analysis was performed by Progenesis Inc (La Jolla, CA) testing laboratory and a next-generation sequencing platform. Following cell lysis and DNA extraction, whole genome amplification was performed on the specimens using the ReproSeq™ PGS Kit. Amplified products were pooled to form the initial library and template enrichment was performed using the Ion Chef automated system. The library was finally loaded on a 540 chip for sequencing, using the Ion S5 XL Sequencing Kit (Life Technologies). There were an average of 150,000 to 200,000 reads and 200 base pairs per amplicon. Filtering for polyclonals was performed with Torrent Suite™ software and then evaluated for aneuploidy using Ion Reporter™ software (Thermo Fisher Scientific). NGS plots were evaluated for chromosome copy number to determine both whole chromosome and segmental aneuploidies.

### Embryo Exclusion Criteria

After fixation, imaging and initial characterization of the 29 embryos that developed to the blastocyst stage, 7 embryos were excluded from further analysis for the following reasons: (1) Four embryos had an abundance of dead cells (both from the GFP+ and GFP− clone) that made it impossible to reliably quantify clonal contributions; (2) Two embryos were completely GFP− and it was impossible to assess whether the initial injection had not been successful (no embryos with only GFP+ cells were observed); and (3) One embryo from the pilot trial had a very small ICM (4 cells).

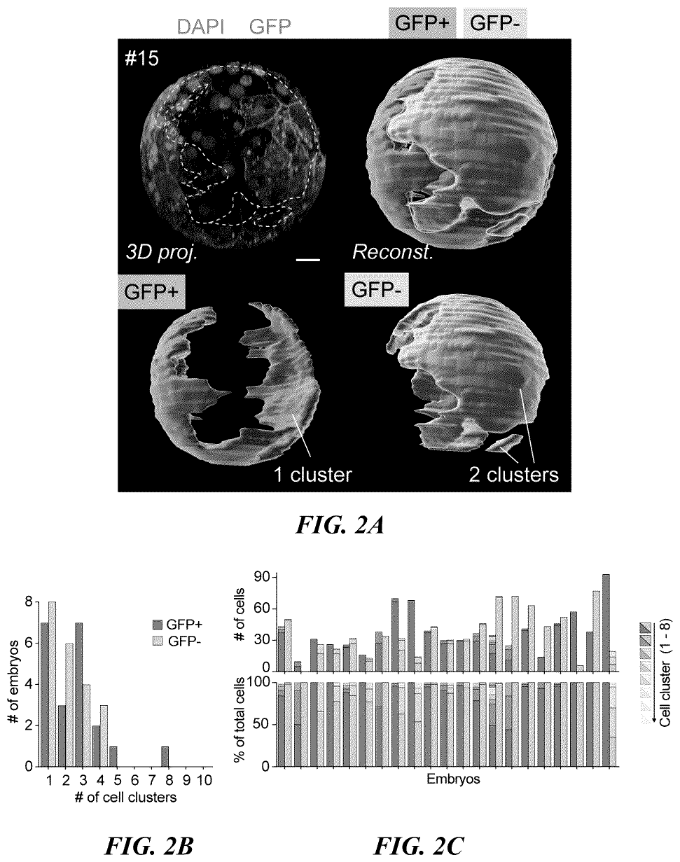

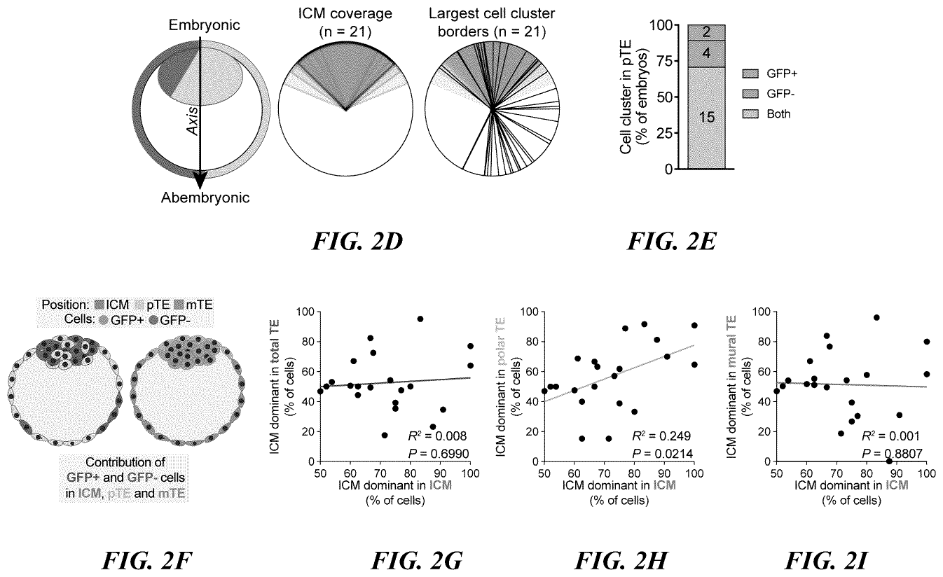

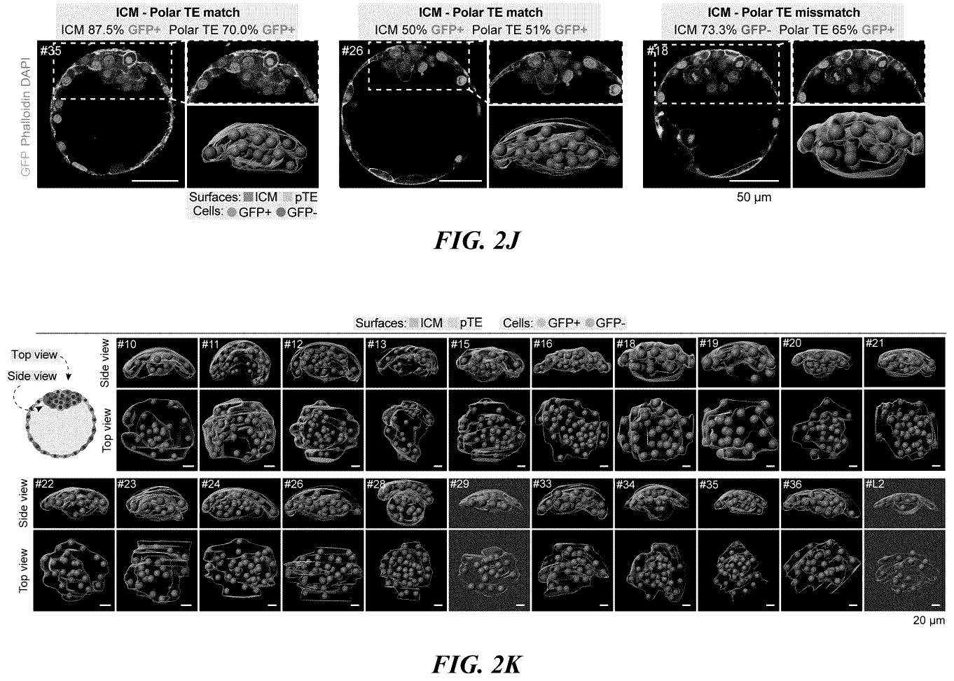

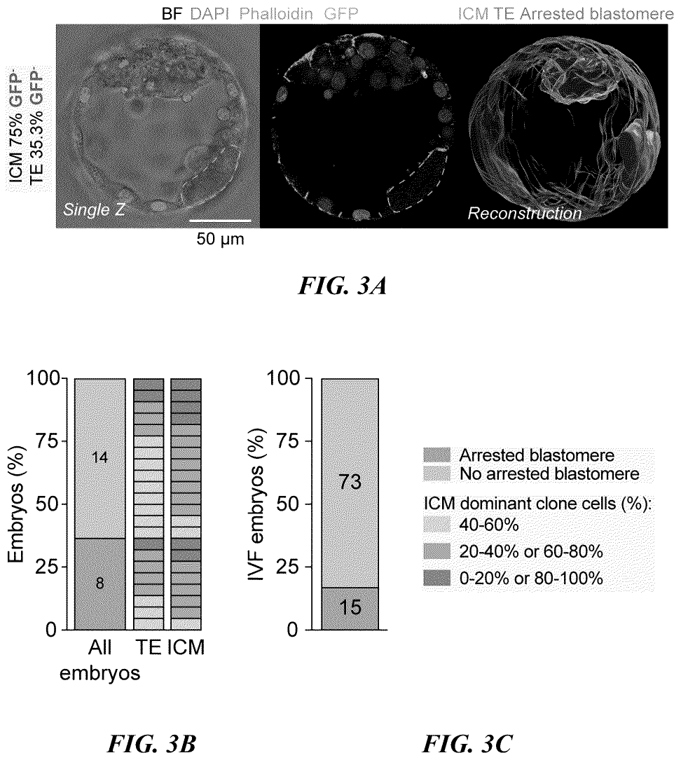

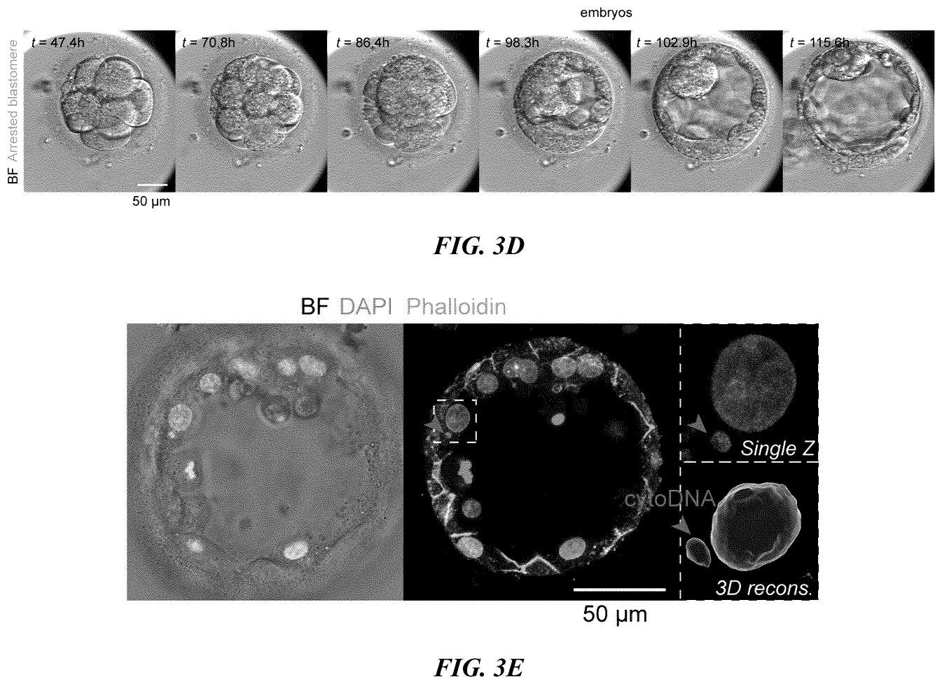

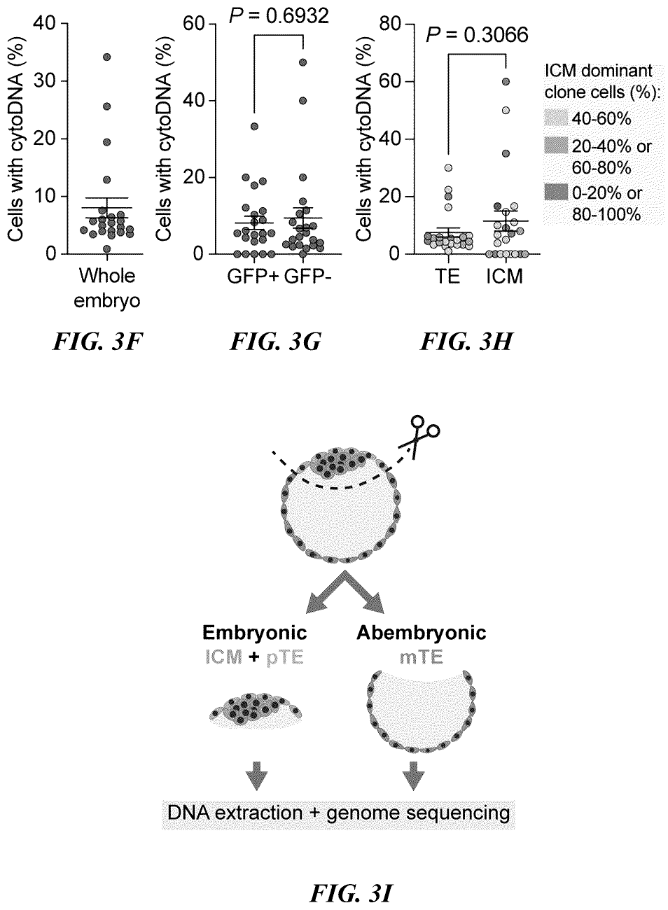

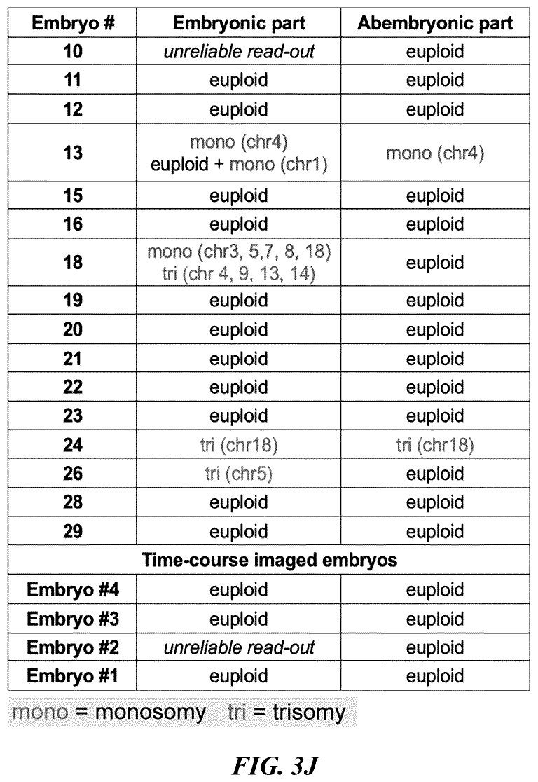

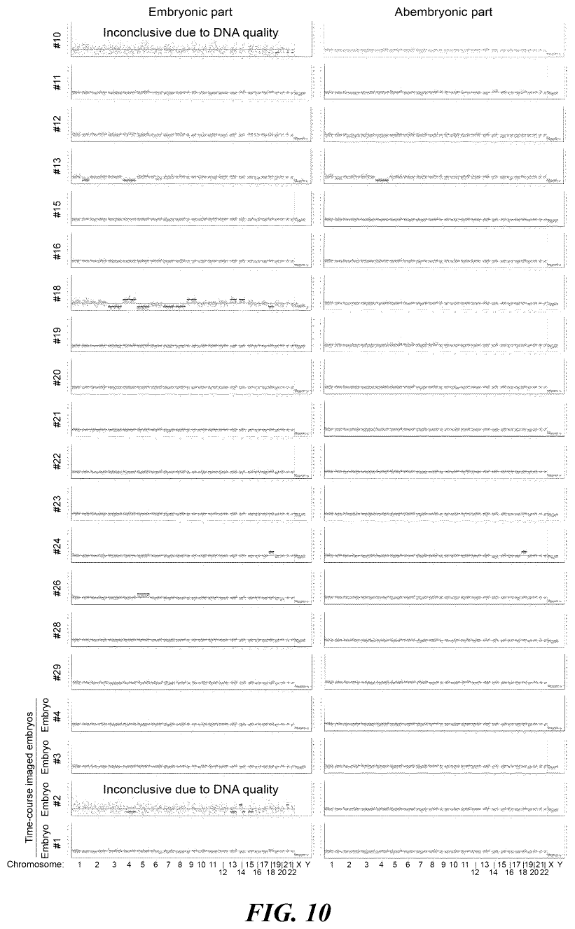

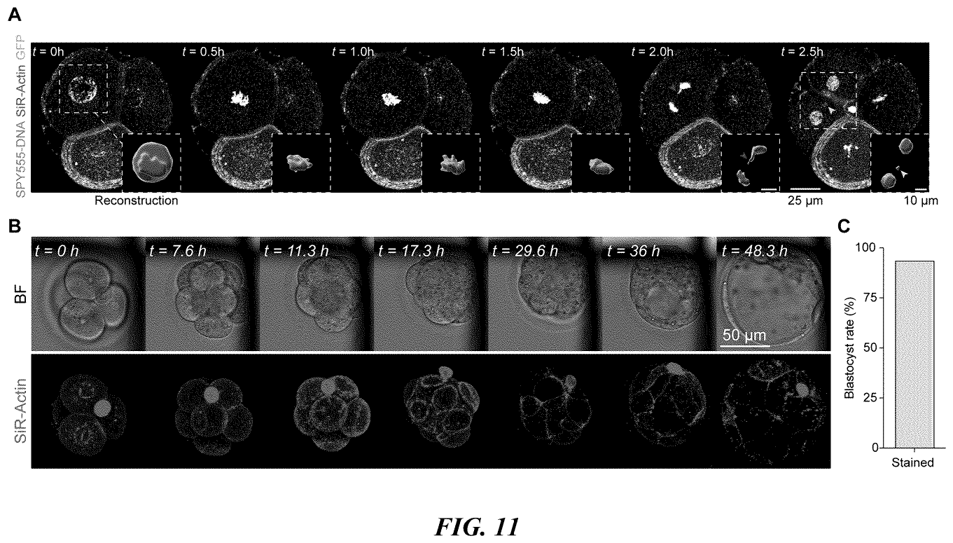

All other embryos (n=22) were used in all reported quantifications. Exceptionally, one or more embryos were excluded from one analysis for reasons particular to the measurements performed. These cases are detailed in the figure legends, and include: (1) Two embryos from the test trial were not accessible for SOX17 staining and were excluded from the quantifications in FIG. 1K, FIG. 8, panels H and I. The sample size for these experiments is n=20; (2) Two embryos from the test trial were not accessible for DNA extraction and karyotyping and were excluded from FIG. 3J and FIG. 10. The sample size for these experiments is n=20; and (3) One embryo was damaged during sample processing and was excluded from the topographical analyses in FIGS. 2B-I. The sample size for these experiments in n=21.

### Number of GFP+ and GFP− Cells in the Human Blastocysts

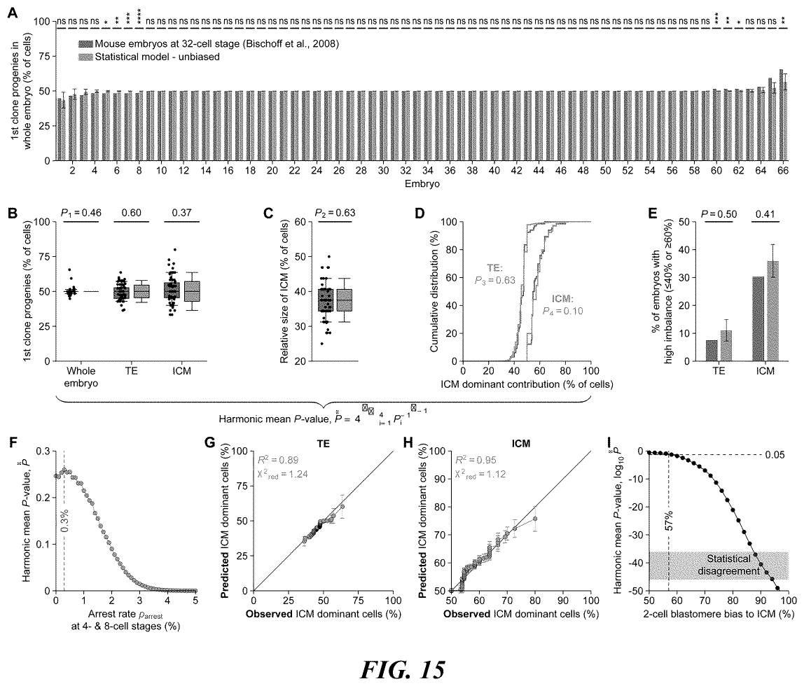

Counting of GFP positive and negative cells in the ICM or TE was completed by 2 independent assessors in a blinded manner, using Fiji (ImageJ). In the case of any discrepancies between these independent assessments, a third assessment was completed by the two assessors together to discuss reasons for the discrepancies and agree on the actual number of cells. GFP intensity was not a relevant measurement in the quantifications performed. Rather, the expression or absence of GFP in a cell was assessed. Therefore, small variations in the level of GFP expression did not have an influence on the results. After injection, mGAP43-GFP mRNA is expressed in the labelled cell and all of that cell's descendants throughout preimplantation development. Extensive internal controls (FIGS. 1A-1J, FIG. 8, FIGS. 3A-3J, and FIGS. 6A-61) were performed that demonstrate that microinjection did not have deleterious effects on the developmental potential of the labelled blastomere.

GFP+ and GFP− cell counts were used to quantify the clonal composition of the TE, ICM, EPI and HYPO, according to the contribution of either cell population to each compartment. The most abundant clone (>50% cells) in the ICM was labelled “ICM-dominant” and used as reference population in both the ICM and the TE. In one embryo, either the GFP+ clone or the GFP− clone could be ICM-dominant.

### Scoring of Blastocysts Using the Gardner and Schoolcraft Blastocyst Grading System

Transmitted brightfield images of blastocysts were analyzed by two independent researchers to score their overall fitness using the Gardner and Schoolcraft blastocyst grading system. This system uses a code of one number plus two letter to describe the expansion level of the blastocyst stage (number; 1 to 6), and the size and aspect of the ICM (first letter; A to C) and the TE (second letter; A to C). A combination of letter scores was used to assign a “Good”, “Fair” or “Poor” quality assessment to each embryo, following this grading: Good (AA, AB, BA), Fair (AC, BB), Poor (BC, CA, CB, CC).

### 3D Projections of Human Blastocysts

Three-dimensional (3D) embryo projections were generated using Imaris software (Oxford Instruments). The “Spots” tool was used to generate a 3D map of the position (ICM or TE) and GFP expression (GFP+ or GFP−) of the cells in the analyzed blastocysts. Spots segmentation was semi-automated, based on DAPI expression, and corrected manually. Classes for position and GFP expression were assigned manually. The “Surface” tool was used to generate 3D volumetric reconstructions. Surface segmentation was performed manually and adapted to the requirements of each measurement (as displayed in the figures). For time-course movies, the membrane and nucleus of single cells at each frame were reconstructed in Imaris. Symmetric divisions were defined as divisions resulting in two cells that remained on the surface of the embryo; asymmetric divisions led to the ingression of one cell into the embryo. For time-course images, the zona pellucida was digitally subtracted from the image for clarity, as indicated in the figure legends.

### Lineage Tracing of the Human Embryo

Imaris was used to trace the position and linage of each cell in the imaged cleavage stage embryos. The “Spot” tool was used to draw a spot at the center of each cell, using the SPY555-DNA signal as reference. Spots for each cell were linked throughout the movie to generate the lineage of that cell, including the time and position (XYZ coordinates) of cell divisions, and the final position of the cells after division. Cell classification, according to GFP expression (GFP+ and GFP−), as well as the final cell position in the embryo (inside or outside) was recorded.

### Angle of Cell Division

For the four cleavage stage embryos imaged, cell division angle was measured using Imaris. For each cell division, at the time of cytokinesis, the X, Y and Z coordinates for the two daughter cells and for the geometric center of the embryo were annotated. The angle formed between the vectors connecting daughter 1-center of the embryo and daughter 2-center of the embryo was measured and used in the figures.

### Number, Position and Borders of Clone Clusters in Trophectoderm

Quantification of the number, position and size of the TE cell clusters was performed using Imaris software. The spot representations of the cells, as well as the actual Phalloidin signal (which reports cell borders) were used for the measurement. A cell cluster was defined as a single cell, or a group of cells of the same type (GFP+ or GFP−) that contacted each other and were fully bordered by cells of the other type. In most embryos, one larger cell cluster for each clone was apparent, containing most of the cells from that clone in the TE. Other smaller clusters were also present, often close to the edges separating the larger clusters from each clone. The number, size and position for each cluster in each blastocyst were recorded and post-processed, as presented in the figures. To describe the spatial organization of GFP+ and GFP− cell clusters, the larger cluster form each clone was used. Using Imaris software, embryos were positioned to place the ICM at the top of the image and the line separating the largest GFP+ and GFP− clusters could be seen clearly. The border location was registered for each embryo as a vector between the center of the embryo and the edges of the cell cluster. The TE area covered by each clonal cell cluster was calculated as the angle formed between the vectors connecting the top of the cluster and the center, and the bottom of the cluster and the center. The clone forming each cell cluster (GFP+ and GFP−) was also annotated.

### Clonal Composition of the Polar Vs Mural TE

Spot representations of the cells, as well as the actual Phalloidin signal (which reports cell borders), were used for the quantification of GFP+ and GFP− cells in the mural vs polar TE. The polar TE region was defined as the TE area containing cells that were in direct contact with the ICM. A surface reconstruction of each polar TE and ICM was prepared using the “Surface” tool on Imaris. These surfaces are included in FIG. 2K. The number of GFP+ and GFP− cells in this area (polar TE) and outside of it (mural TE) were then calculated. Data comparing the contribution of the ICM dominant population in the ICM and the contribution of the ICM dominant population in either the polar or the mural TE are presented.

### Cell Cycle Length

Movies of SiR-Actin and SPY555-DNA stained or unstained mouse embryos were used to assess the effect of dye staining on the division rate. The time at which each cell completed cytokinesis at the 4- to 8-cell division stage was recorded. Additionally, the time at which each cell completed cytokinesis at the 8- to 16-cell division was also recorded. Both values were used to measure the length of the 8- to 16-cell cycle, presented in the figures.

Movies following SPY555-DNA and SiR-Actin-stained embryos during the 8- to 16-cell transition were used to measure the duration of interphase and mitosis. For each cell, the time at which cytokinesis was completed at the 4- to 8-cell stage division was recorded. Furthermore, individual cells were observed to record the time at which the major phases of mitosis could be seen (prophase, metaphase, anaphase, telophase and cytokinesis).

Lineage Tree Reconstructions from 1- to 16-Cell Stage Using Embryoscope™ Time-Lapse Movies

Embryoscope™ time-lapse movies capturing preimplantation development in human embryos from the zygote to blastocyst stage were provided by IVIRMA-Valencia (IVI Foundation, Spain). Each movie contained transmitted light images at 11 focal planes with an average imaging frequency of 15 minutes. All samples represented embryos that resulted in successful pregnancies and live births. Manual tree curation and division scoring was performed by two independent researchers, in two locations in a blinded manner using TrackMate. Division scoring was done before tree curation so that the assessors remained blinded to the clonal assignment of the assessed divisions. The following parameters were recorded: timing of division of each blastomere at 2-, 4- and 8-cell stage, clonal history of each cell, symmetric/asymmetric cell division history of each cell during 8- to 16-cell transition. It was not possible to trace cells and lineage fate beyond the 16-cell stage, due to the loss of single cell resolution on compaction. Fifty-four movies were of sufficient quality to annotate all required parameters with confidence and in agreement between the annotators.

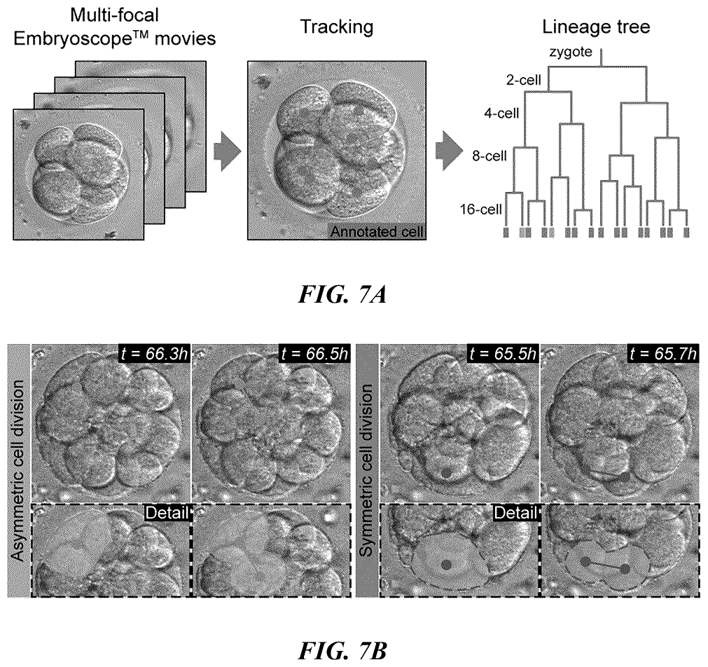

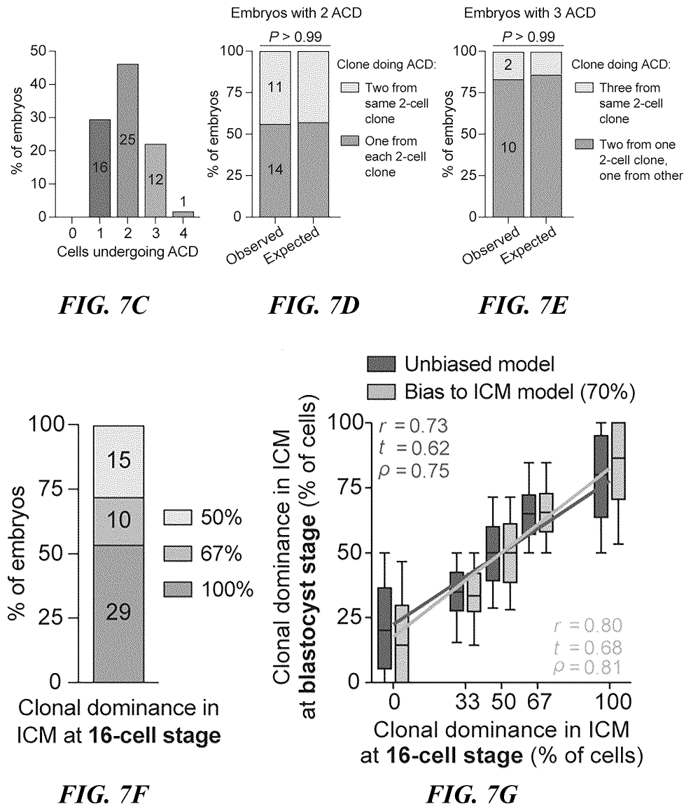

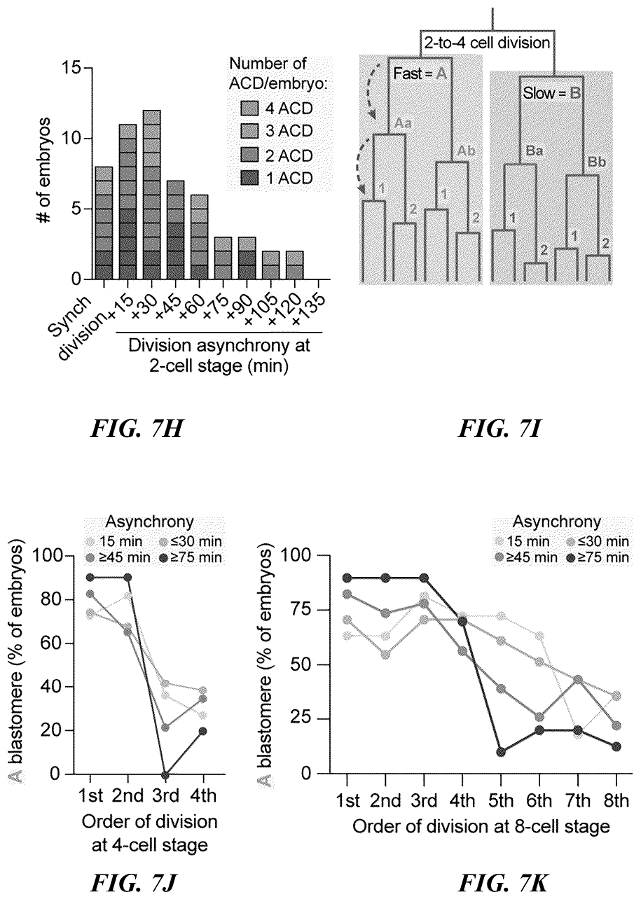

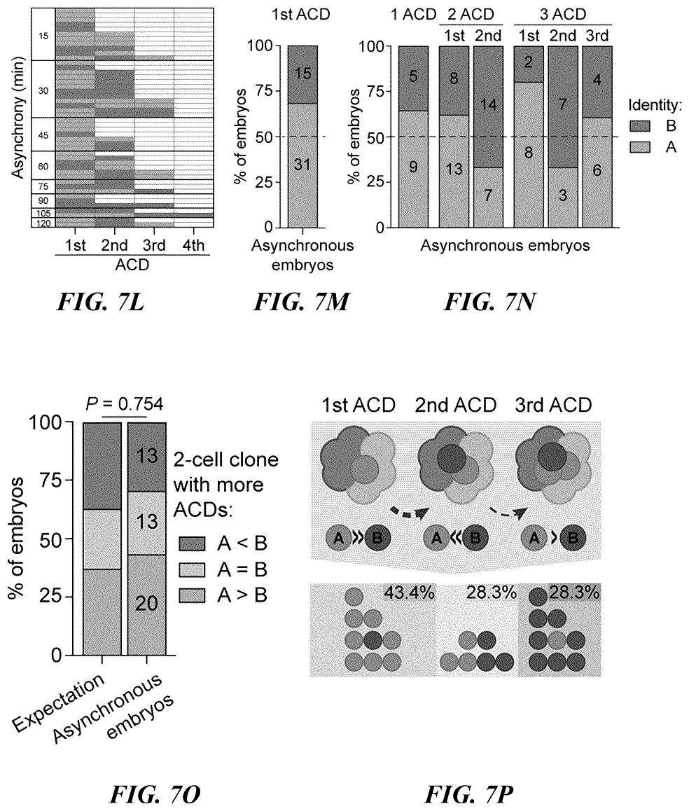

Expected proportions in FIGS. 7D, E and O were calculated using the probability mass function of the hypergeometric distribution (PMFHD):

$$P ( x , M , K , N ) = \frac{( \begin{matrix}K \\ x\end{matrix} ) \times ( \begin{matrix}M - K \\ N - x\end{matrix} )}{( \begin{matrix}M \\ n\end{matrix} )}$$

where x is the number of ACDs (2 or 3) done by the same 2-cell clone, M is the number (8) of mother cells, K is the number (4) of same-clone mother cells and N is the number (2 or 3) of total ACDs per embryo.

PMFHD assumes that the outcome of one event modifies the probability of the subsequent events (e.g. If in one embryo with 2 ACDs the first cell that undergoes an ACD originates from the faster “A” 2-cell blastomere (P=4/8 or 50%), the probability for the second ACD to be from the “B” clone is higher (P=4/7 or 57.1%) than of that of it being from the “A” clone (P=3/7, 42.9%)). For the expectation in FIG. 7O, the fraction of total (n=46) cases in which a distribution of embryos with 1, 2, 3 or 4 ACDs like the one measured in the sample (FIG. 7C) would result in embryos with A>B, A=B or A<B inside cells was calculated. This is presented in the figure.

### Detection of the Presence of Arrested Blastomeres

Blastocyst images generated in this study were used to detect the presence of large arrested blastomeres at the surface of the TE. For this, brightfield images were used, and large cell remnants that appeared arrested were quantified. Embryoscope™ time-course brightfield images of embryos developing from the zygote to the blastocyst stage were provided by IVIRMA-Valencia (IVI Foundation) (as described above) and were used to measure the number of embryos with arrested blastomeres. Movies were visually inspected using ImageJ and the presence of abnormally sized blastomeres with a darker or oddly textured appearance that arrested and did not divide further was recorded, as well as the number of embryo cells present at the time of the first appearance of such structures. Eighty-eight embryoscope movies were successfully analyzed for the presence of arrested blastomeres between the 2- and 16-cell stage.

Statistical Model of Blastocyst Development with Random Cell Arrest, Death and Internalization

A Markov Chain Monte Carlo model of blastocyst development was developed to study the distributions of GFP+ and GFP− cells in the whole embryo and in the ICM/TE in detail, ignoring geometrical or other spatial effects (with an exception detailed below). Using this model, pools of 22 embryos were generated. Each embryo starts at the two-cell stage, with one blastomere marked by the GFP, and the other unmarked. The embryos then undergo consecutive rounds of cell division, during which the cell type (GFP+ or GFP−) is inherited by the daughter cells. At the 4-cell and 8-cell stages, a random subset of the blastomeres was let arrest (i.e., not divide any further), and from the 64-cell stage onward another random subset (possibly including already arrested cells) was let die. Both cell selection processes for arrest and death are assumed to be independent Bernoulli processes with probabilities pₐᵣᵣₑₛₜ and p_{death} in each affected cleavage cycle. Thus, the fraction of blastomeres undergoing arrest or death is not fixed at pₐᵣᵣₑₛₜ and p_{death}, but varies across these probabilities according to a binomial distribution. Cell arrest and death are assumed to occur to GFP+ and GFP− blastomeres, as well as to those in the ICM and TE, with equal chance.

In the absence of detailed information regarding non-uniformity in cell death over space and time, other than that it is observed primarily after the morula stage, cell death is assumed to occur only from the 64-cell stage onward at a constant rate p_{death}. In some embodiments, p_{death}=4.4% such that the average percentage of dead cells at the blastocyst stage lies in the middle of the previously reported range of 7-8% (FIG. 13, panel A). Meanwhile, the selected rate of cell arrest pₐᵣᵣₑₛₜ=6.5% is based on a maximization of the statistical agreement between the model prediction and the data (FIG. 13, panel B), as quantified by the harmonic mean P-value from four two-sample Kolmogorov-Smirnov tests that compare the clonal distributions across the whole embryos, the distributions of relative ICM sizes, and the distributions of the shares of the ICM dominant clone in the TE and ICM (FIGS. 6C-E):

$$P ° = \frac{4}{{\sum }_{i = 1}^{4} 1 / {P}_{i}} .$$

The identified optimum of 6.5% that maximizes P^{o} lies well in the range of the previously reported rates of 4.3% and 8.3% for the 4- and 8-cell stages⁴³.

After the 3^{rd}, 4^{th} and 5^{th} round of cell division (i.e., during the 8- to 16-, 16- to 32- and 32- to 64-cell stage transitions), a subset of the daughter blastomeres was randomly selected and internalized, generating a potential lineage imbalance. From this stage onward, the ICM and TE cell pools continue dividing separately. The number of cells internalizing, nᵢₙₜₑᵣ, like the arrest and death rates, is not fixed, but drawn uniformly within a predefined range [nₘᵢₙ, nₘₐₓ] in each cleavage generation. Based on the observations in human embryos, nₘᵢₙ was set to 1,0,0 and nₘₐₓ to 3,2,1 in the three cleavage rounds with cell internalization, and zero before and after. This approximately reproduces the observed final fraction of ICM cells as 17.4% of the total cells on average (FIG. 6D). Only blastomeres with distinct mother cells are eligible for internalization, to mimic asymmetric division in which just one out of the two sister cells internalizes per cycle. A fourth model parameter, the fate determination bias b, controls the average ratio between internalization of GFP+ and GFP− cells. For unbiased selection (equal average likelihood for GFP+ and GFP− blastomeres to internalize), b=0.5, whereas b=1 represents exclusive internalization of one of the two cell types. The biased average internalization numbers are thus given by bnᵢₙₜₑᵣ and (1−b)nᵢₙₜₑᵣ for the respective cell types.

Motivated by a potential buffering role of geometric constraints on the number of internalizing blastomeres, the model additionally includes a “memory effect” to partially compensate in subsequent cycles for relatively few or many asymmetric cell divisions in previous cycles. The deviation in the cumulative expected mean number of internalized blastomeres from the actually realized cumulative sum until generation i,

$${\sum }_{j = 3}^{i} \frac{{n}_{min , j} + {n}_{max , j}}{2} - {n}_{i n t e r , j} ,$$

is added to the next number of internalizing cells, n_{inter,i+1}. Since cell numbers approximately double in each generation, the impact of this compensation on the size of the ICM halves in each generation.

Once each embryo reaches the final number of blastomeres as observed in the human dataset, they are sorted by their contribution of GFP+ cells. Repeating this stochastic process 10⁴ times allows us to evaluate the statistically expected distribution of GFP+ and GFP− cells in the whole embryos, and in the ICM and TE individually.

The clonal composition was recorded at early stages, for example after the first internalization wave at the 16-cell stage. The clonal composition at early stages was then compared to the final outcome at the blastocyst stage, to study the importance of early ACDs. It is observed that an existing clonal imbalance at the 16-cell stage is a strong predictor of the clonal dominance in the ICM at the blastocyst stage in both the unbiased and biased models (FIG. 7G). In 87.5% of all 220,000 simulated embryos without fate bias (89.9% for the model with 70% bias), the embryos with a clonal imbalance at the 16-cell stage developed into blastocysts with the same clone dominating the ICM.

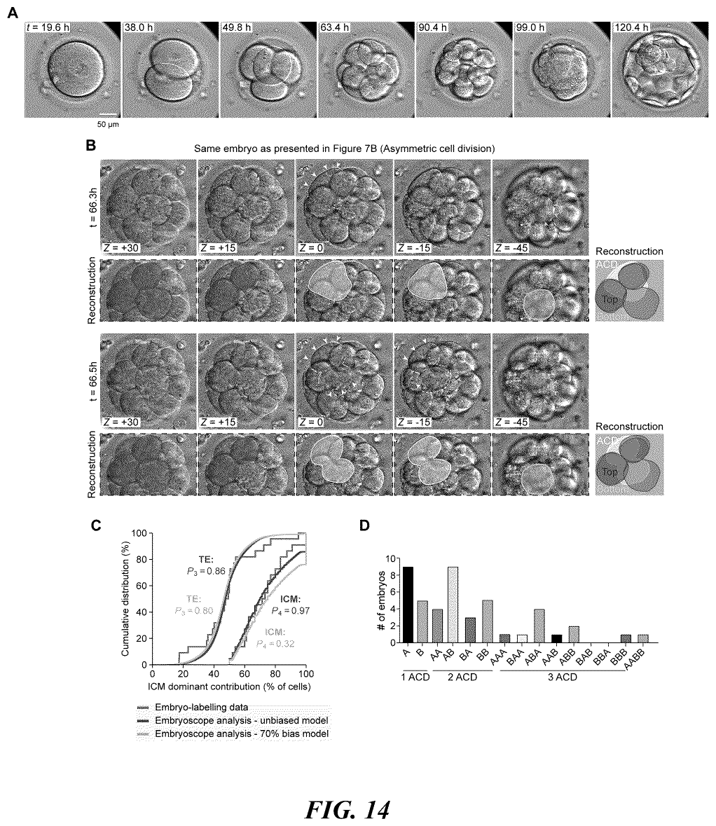

To verify whether there are any statistically significant differences in the two human embryo datasets that were used in this study, a subset of the simulated embryos that had the exact clonal compositions at the 16-cell stage as those observed in the Embryoscope™ movies was used, and the blastocysts they develop into according to the biased and unbiased models was compared to the blastocysts observed in the labelled dataset (FIG. 14, panel C). No statistically significant differences were found in the compositions of the TE or ICM of the resulting blastocysts.

Finally, the model is validated against a published dataset that tracked mouse blastomeres in embryos from the 2 to 32-cell stages. The model that best recapitulates the published data is one with no fate bias (b=0.5), nᵢₙₜₑᵣ=1 in the 1^{st} internalization wave (8 to 16 cells), and nᵢₙₜₑᵣ=8-12 in the 2^{nd} wave (16 to 32 cells). The corresponding P-values analogous to FIG. 6 are P₁=0.46, P₂=0.63, P₃=0.63, P₄=0.10, indicating that the model for human embryo development is also capable of reproducing early mouse embryogenesis within the range of statistical errors and with appropriately adjusted numbers of ACDs (Supplementary information).

P-values of statistical comparisons were either directly evaluated from the results of the 10⁴ model realizations (as the probability of the model predicting values at least as extreme as observed in the data), or calculated with two-tailed Kolmogorov-Smirnov tests, as indicated in the figure captions. Custom MATLAB code has been deposited and can be found here: git.bsse.ethz.ch/iber/Publications/2024_junyent_meglicki_blastomeres.

### Example 2

### Lineage Tracing of Human 2-Cell Stage Blastomeres

Retrospective tracking of somatic mutations in the human body and placenta predict clonal imbalances may arise as early as the 2-cell stage of the embryo. To determine whether this is indeed the case, this example aimed to track forward the lineages of early stage blastomeres of human embryos for the first time. Such prospective studies of human development are challenged by limited availability of human embryos at the zygote stage. Nevertheless, 54 human in vitro fertilized zygotes that had been donated for research were obtained for this investigation.

First, a neutral lineage reporter was used to follow progeny of individual 2-cell blastomeres through their development to the trophectoderm (TE) and inner cell mass (ICM) of the human blastocyst (FIG. 1A). Briefly, human zygotes were thawed and cultured until the completion of the first cleavage division. At the late 2-cell stage (approximately 17 h post-thawing), one random blastomere was microinjected with mRNA encoding GFP fused to the membrane targeting sequence of GAP43 (i.e. not full length, mGap43-GFP; Methods), which has been reported to not affect embryo development. mGap43-GFP marked the boundaries of injected blastomere and its progeny, allowing us to discern lineages from each 2-cell blastomere as GFP-positive (GFP+) versus GFP-negative (GFP−).

Embryos were cultured for 4-5 days and fixed as expanded blastocysts, which were stained with AF647-Phalloidin to detect F-actin networks and DAPI to detect chromatin. Blastocysts displayed mosaic GFP expression throughout the TE and the ICM (FIG. 1B, FIG. 8, panel B) and had a healthy morphology (FIG. 8, panels C-E). 97.3±27 [mean±SD] cells were found in the whole embryo, 80.8±24 cells in TE and 16.5±7 cells in ICM, which comprised 11.8±5 epiblast (EPI) cells and 5.2±3 hypoblast (HYPO) cells (FIG. 8, panels F-H), as expected. The number of HYPO cells positively correlated with overall ICM cell number (FIG. 8, panel I), as expected. Each of the 22 analyzed blastocysts contained GFP+ and GFP− cells that contributed to both TE and ICM (FIGS. 1C-D, FIG. 8, panel J). These results show that the lineage tracing does not affect human development or the developmental capacity of the injected blastomere.

### Example 3

### 2-Cell Stage Human Blastomeres Contribute Unequally to ICM, Epiblast, and Polar TE

To determine the contribution of each 2-cell stage blastomere to all three lineages, the percentage of GFP+ and GFP− cells in each lineage was quantified. The mean frequency of GFP+ cells in the population was ˜50% in the whole embryo (50.8% or 48.8±19 cells) as well as the TE and ICM (50.3% or 36.3±22 cells in TE and 49.4% or 7.4±6 cells in ICM) (FIGS. 1C-D). The differential GFP labeling allowed us to discern the dominant (contributing >50% of cells) and non-dominant populations in the ICM and TE of each blastocyst (FIG. 1E). In the ICM, the dominant population accounted for, on average, 71.25% of cells (11.7±5 cells). In contrast, the TE-dominant population accounted for, on average, 62.86% of cells in the TE (51±19 cells) (FIG. 1F). The dominant population in either compartment was GFP+ or GFP− with near-equal chance (FIG. 8, panel K), confirming that GFP expression did not affect blastomere development.

The contribution of each 2-cell clone to the ICM and TE in each individual embryo was then investigated (FIGS. 1G-I). Both 2-cell blastomeres contributed near-equal amounts of cells to the ICM in only 3/22 blastocysts (13.7%) whereas a single 2-cell clone contributed between 60-100% of ICM cells in 19/22 embryos (86.3%) pointing to a clonal imbalance in their ICM (FIGS. 1G-H). 10/22 embryos (45%) displayed a clonally imbalanced TE, with 4/22 embryos having ≥60% ICM and TE cells originating from the same 2-cell blastomere (FIGS. 1G-H, FIG. 8, panel L). On average, the ICM-dominant clone, which represented 71.25% of ICM cells, represented 49.36% of the TE (38.7±19 cells) (FIG. 1I). These clonal imbalances were independent of the embryo size (Sup. FIG. 1M-P) and embryo quality (FIG. 8, panel E). Importantly, it was found that within the ICM, one 2-cell blastomere generated an average of 76% of epiblast cells (average 9±4.1 out of 12±5.3 EPI cells) and 53.7% of hypoblast cells (average 2.5±1.4 out of 5.2±3 HYPO cells) (FIGS. 1J-L).

In the TE, most embryos displayed 1-3 cell clusters of GFP+ or GFP− cells (FIGS. 2A-C), with the largest clusters spreading along the embryonic-abembryonic axis (running meridionally from the pole having ICM to the opposite pole of the blastocyst, FIGS. 2D-E, and FIG. 9). Thus, cells derived from each 2-cell blastomere contributed to both polar TE (overlaying the ICM) and mural TE (covering the ICM-free blastocoel, FIG. 2E). Importantly, the clonal composition of the ICM did not correlate with that of the mural TE or the whole TE, but did positively correlate with the polar TE composition (FIGS. 2F-K).

Taken together, asymmetries in the contribution of a 2-cell blastomere to the ICM versus TE and to the epiblast versus hypoblast within the ICM was detected.

### Example 4

### 2-Cell Clonal Imbalance is not Explained by Blastomere Arrest or Genomic Instability

To investigate how this lineage bias arises, parameters of embryo quality were first examined. Large, mitotically arrested blastomeres in 8/22 blastocysts (36.4%) (FIG. 3A) were observed, whose size suggested they arrested at the 4- or 8-cell stage, while the remaining blastomeres progressed in development. However, there was no clear correlation between clonal composition of the TE or ICM and the rate of blastomere arrest (FIG. 3B), indicating that blastomere arrest does not underpin the observed clonal imbalances. The rate of blastomere arrest in embryos freshly generated by in vitro fertilization (IVF) was also measured and recorded with the Embryoscope™ system in the IVF clinic (Methods). It was observed that 15 out of 88 such embryos (17%) also had cells arresting around the 8-cell stage (FIGS. 3C-D).

Many human embryos are thought to contain aneuploid blastomeres, which could lead to miscarriage or congenital defects³⁰⁻³³. In mouse embryo models of mosaic aneuploidy, aneuploid clones can be cleared from the ICM lineages but survive in the TE, albeit with proliferative defects. Whether a similar clearing mechanism exists in the human embryo remains contested, but its existence has been suggested to explain lineage allocation biases in the early embryo. Time-lapse imaging of human embryos revealed the presence of divisions with “lagging chromosomes”, a hallmark of chromosomal instability. In these blastomeres, chromosome separation at anaphase was incomplete and resulted in cytoplasmic DNA clusters (cytoDNA) (FIG. 11, panel A). On average, cytoDNA was observed in 8.1% of cells of the 22 blastocysts. CytoDNA appeared at similar rates in both GFP+ and GFP− cells, as well as ICM and TE cells and the proportion of cytoDNA-containing cells was no different between embryos having different degrees of 2-cell clonal asymmetry (FIGS. 3E-H). This indicates that chromosome instability is not a primary driver of the observed clonal imbalances.

Given that ICM clonal composition correlated with the polar but not mural TE (FIGS. 2H and I), this example also aimed to identify potential karyotypic differences correlating with the observed clonal imbalances. ICM and polar TE were isolated from mural TE for sequencing (FIG. 3I). Of 20 embryos sequenced, 2 gave partially inconclusive results; 14 were completely euploid; 2 were completely aneuploid; and 2 were aneuploid only in the ICM and polar TE (FIG. 3J, FIG. 10). Overall, only a small subset of the analyzed embryos had aneuploidies suggesting that clonal imbalances are independent of karyotypic aberrations.

Together with internal quality controls (FIGS. 1A-1J, FIG. 8), these results argue against a primary role for blastomere arrest and aneuploidy in modulating the clonal composition of the blastocyst.

### Example 5

### Live Imaging Reveals Asymmetric Cell Division at the 8-Cell Stage Predict ICM Composition

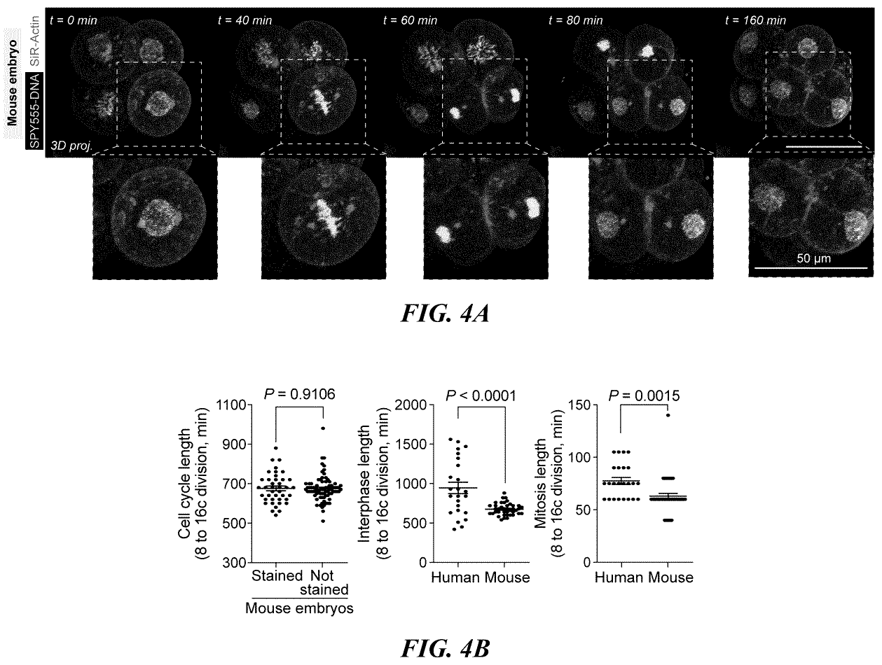

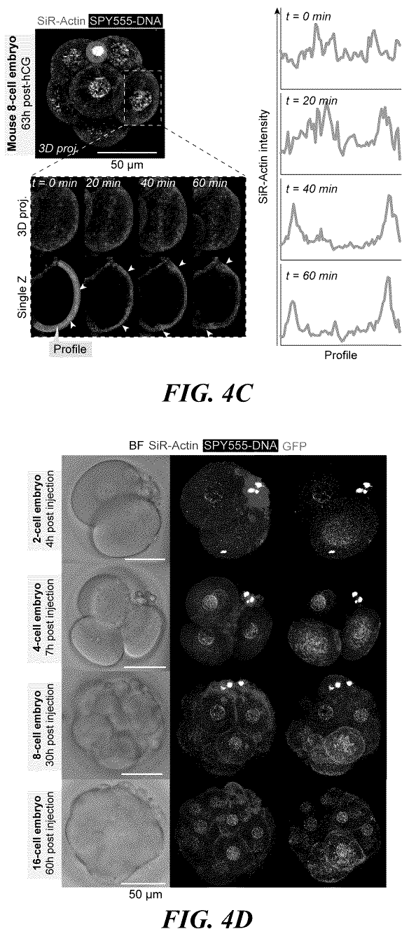

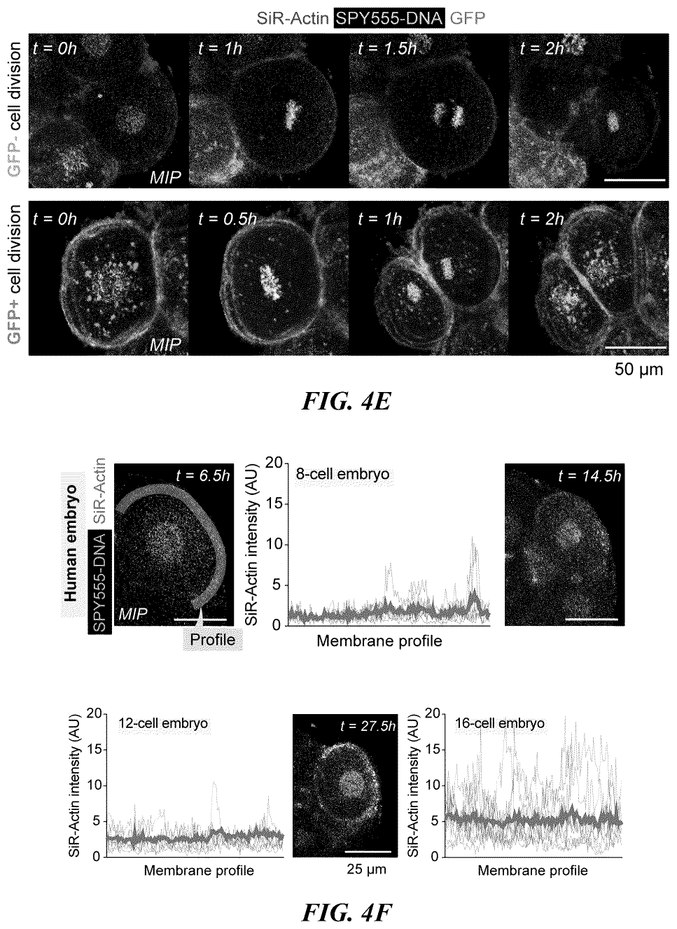

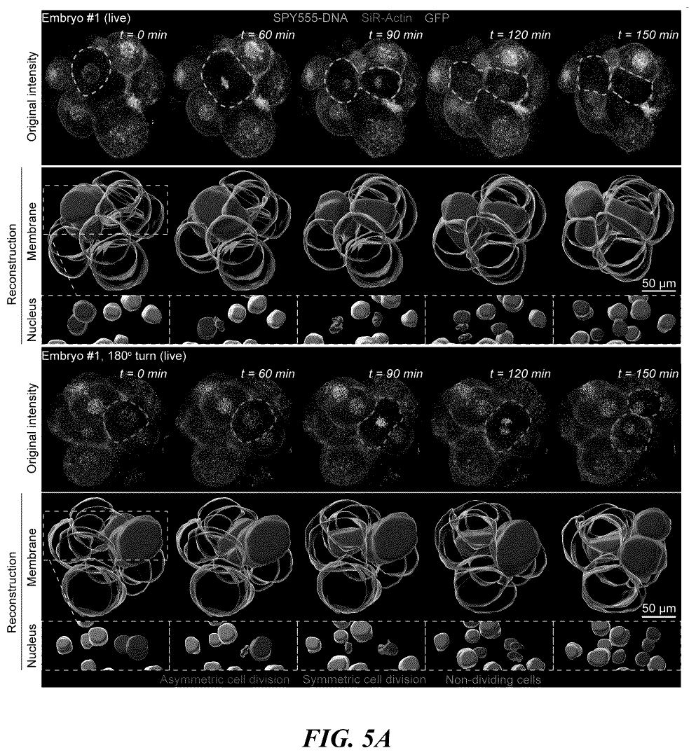

In the mouse, ICM cells are allocated through three successive rounds of asymmetric cell division (ACD) from the 8- to the 64-cell stages. The first wave of ACD at the 8- to 16-cell transition is biased towards generating epiblast cells, whereas the second and third waves of ACD generate primitive endoderm (equivalent to human hypoblast). ACD dynamics in the 8-cell stage human embryo are poorly understood, and clonal imbalances in ICM cell allocation could be important in controlling ICM and epiblast composition. To monitor ACD in human embryos, membrane-permeable fluorescent dyes were used to track genomic DNA (SPY555-DNA) and F-actin (SiR-Actin) in live imaging experiments (FIG. 4A). As a proof-of-concept, 2-cell stage mouse embryos were recovered from pregnant females, stained, and imaged until the blastocyst stage (FIG. 4A, FIG. 11, panel B). The cell cycle durations and rate of progression to the blastocyst stage were similar in stained and unstained mouse embryos (FIG. 4B, FIG. 11, panel C). In addition, SiR-Actin staining allowed us to quantify the formation of the cortical F-actin ring in the apical region of 8-cell stage blastomeres (FIG. 4C).

The live embryo labelling protocol was then applied to a subset of mGap43-GFP-injected human embryos (FIGS. 4D and E). Nuclear and membrane co-labelling allowed us to follow complete mitotic progression in GFP+ and GFP− human cells (FIG. 4E). Compared to mouse 8-cell blastomeres, human 8-cell blastomeres had a longer interphase (15.75 h in human, 11.24 h in mouse) and mitosis (FIG. 4B). The enrichment of cortical F-actin was also observed during 8- to 16-cell polarization of human embryos (FIG. 4F). Thus, this labelling approach allowed detailed capture of morphogenetic events by 3D confocal, time-lapse imaging.

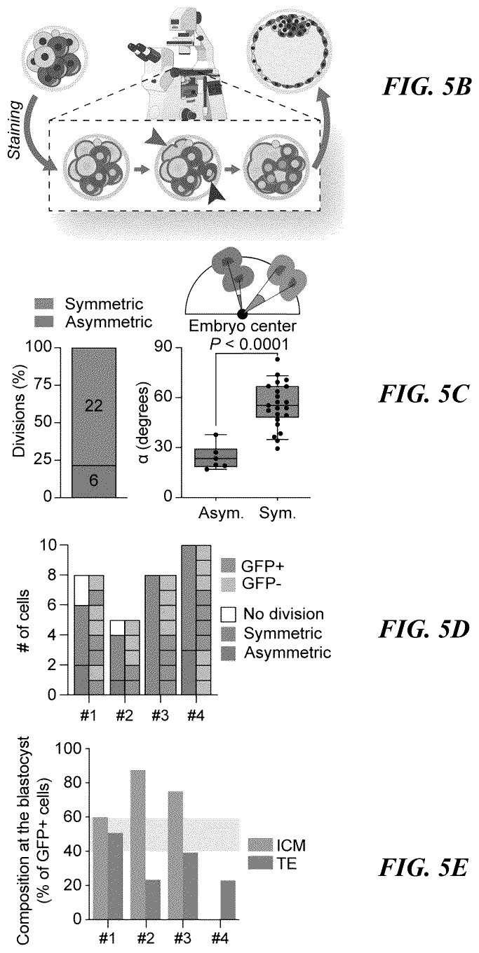

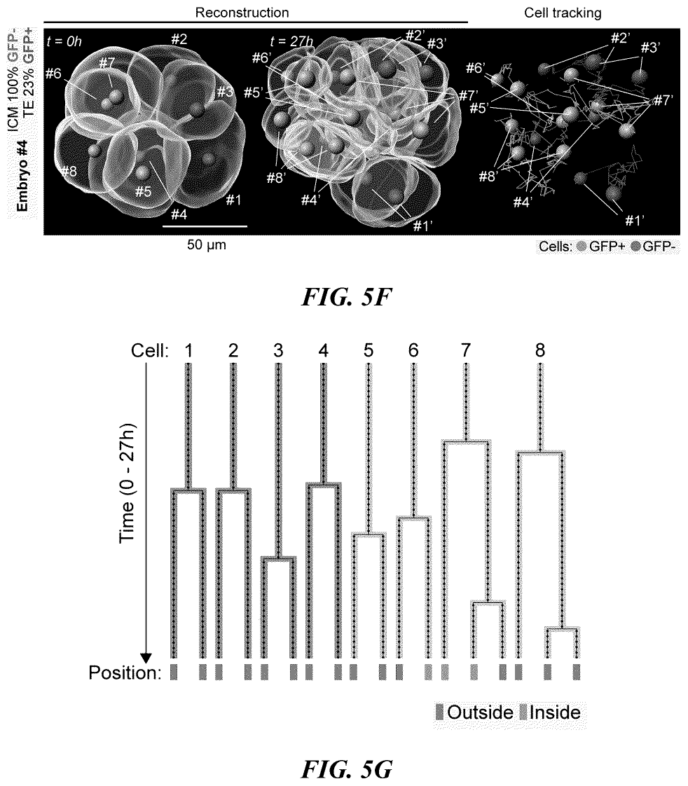

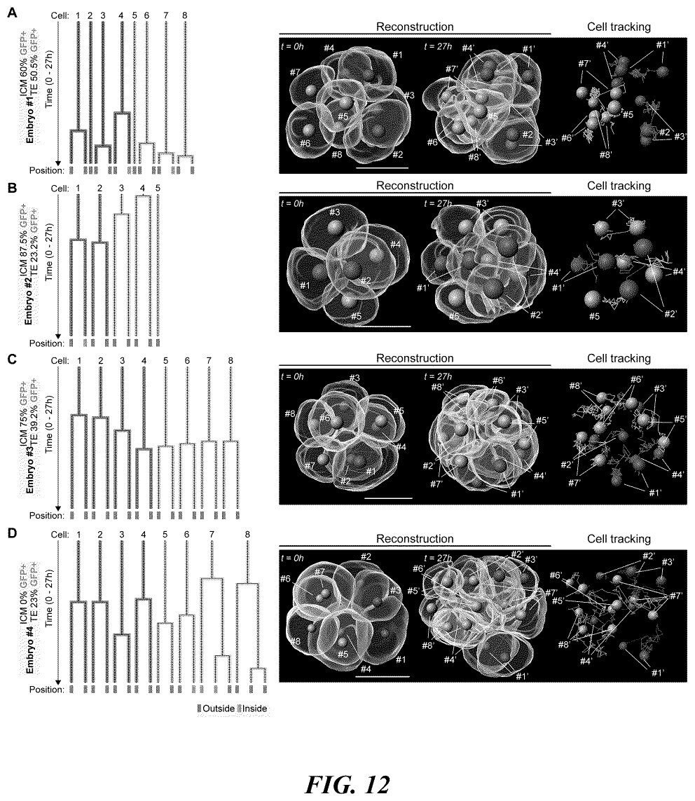

To evaluate ACD at the 8- to 16-cell transition, embryos were stained with SiR-Actin and SPY555-DNA at 27 h post-injection and imaged for a further 28 h (FIGS. 5A-B). The position and division of each cell were tracked over time (FIG. 12). A division is defined as ACD if it led to the ingression of one daughter cell to allocate an ICM cell (FIGS. 5A, C-D, blue cell). In contrast, symmetric cell divisions (SCD) had both daughter cells remaining at the embryo surface (FIGS. 5A, C-D, red cell). ACD and SCD were verified by measuring the angle of division of the cells (FIG. 5C). A total of 28 divisions were observed in the four embryos (FIG. 5D). ACDs were less frequent than SCDs (FIGS. 5C-D), consistent with observations in mice. Importantly, the clonal composition of the ACD strongly predicted the clonal composition of the ICM at the blastocyst stage (FIGS. 5D-E). For instance, in embryo #4, three GFP− cells underwent ACD and the blastocyst ICM was completely occupied by GFP− cells (FIGS. 5F-G). In contrast, in embryo #1, one GFP+ and one GFP− underwent ACD, and GFP+ cells represented 60% of the cells in the blastocyst ICM (FIG. 12, panel A). In embryo #2, one GFP+ cell underwent ACD when the embryo had only 7 cells, and 75% of blastocyst ICM cells expressed GFP (FIG. 12, panel B). Although limited by the small sample size, the results indicate that early cell ingression to the ICM is a strong predictor of ICM clonal composition.

### Example 6

### A Computational Model of Three Parameters Predicts Clonal Asymmetry in Human Blastocysts

To test parameters that could explain the observed clonal composition in human embryos, a statistical model of blastocyst development was developed, which predicts cell distributions through computer simulations. The model generated pools of 22 embryos, each with the total number of cells observed in the human blastocysts, but varying proportions of ICM and TE, averaging 17.4% of embryo cells in the ICM. Each modeled embryo started at the 2-cell stage with one blastomere randomly marked by GFP expression and followed embryo development through successive rounds of divisions. The expression of GFP (or lack thereof) was inherited by the daughter cells (FIG. 6A).

First, three parameters that could affect the clonal composition of the human blastocysts: cell death, cell arrest, and ACD were modulated (FIG. 6A). Based on literature, the model stipulated that death randomly affected any embryo cell from the 64-cell stage onwards, at an average rate of p_{death}=4.4%. This resulted in an average of 7-8% dead cells per blastocyst (see below; FIG. 13, panel A), consistent with Brison, D. R. (2000) (Apoptosis in mammalian preimplantation embryos: Regulation by survival factors. Hum Fertil 3, 36-47. 10.1080/1464727002000198671). A cell arrest rate (pₐᵣᵣᵣₑₛₜ) of 6.5% affecting any cell at the 4- or 8-cell stage regardless of its clonal identity (FIG. 13, panels B-C) was calculated. These assumptions, which are consistent with previous reports, resulted in a final blastocyst pool with the same proportion of embryos with arrested blastomeres as the one measured in the original sample (FIG. 3B, FIG. 13, panel D).

Finally, the model stipulated that each embryo underwent three consecutive waves of ACD, similar to the mouse embryo. Based on calculations from the embryo dataset, it was estimated that the number of ACD in each wave, nᵢₙₜₑᵣ, varied uniformly in the ranges of 1-3, 1-2 and 0-1 cells per wave, respectively (FIG. 13, panels E-F). Interestingly, these values are lower than those measured in mouse, but similar to those estimated in human embryos. Modulating the number of internalization waves (FIG. 13, panel F) or the number of ACD in each wave (FIG. 13, panels E, G) showed that the design was the best at recapitulating the values measured in the blastocysts.

GFP+ and GFP− cells were randomly picked for internalization during these cleavage cycles, a choice referred to as “unbiased” fate determination. For this process to represent both SCD and ACD, only up to one of two sister cells was allowed to be selected for internalization. In the mouse embryo, the number of cell internalizations is compensated across the internalization waves to balance the final number of ICM cells: if too few cells are internalized in the first wave, a higher number of cells will be internalized in the subsequent wave. To model this tendency, this effect was mimicked by automatically adjusting hinter by the difference between the expected number of internalized cells and that accumulated over previous waves. From the 64-cell stage onwards, the ICM and the TE were modeled to divide separately, until the final embryo size was reached.

Using these parameters through 10⁴ repetitions predicted 220,000 embryos with distributions of GFP+/GFP− cells in the whole embryo that were not statistically different from the distributions observed in human blastocysts (FIGS. 6B-C, red). The model corroborated that GFP expression does not affect the developmental potential of injected blastomeres (as both modeled and real embryos had a similar distribution of GFP+ cells, FIGS. 6B-C) and reproduced the observed distribution of ICM sizes (FIG. 6D) as well as the clonal composition of TE and ICM (FIGS. 6E, H, I). This model predicted a proportion of embryos with clonal imbalances in the TE and ICM that was statistically consistent with the in vitro measurements in blastocysts, albeit with lower frequencies (FIG. 6F, red bars). Quantitatively, the cumulative distribution (FIG. 6E) predicted ˜73.1% of embryos with ≥60% of ICM cells from one 2-cell blastomere, and ˜31.8% of embryos with ≥80% of ICM cells from one 2-cell blastomere. By assessing lineage compositions in 220,000 embryos modeled with minimal parameters, the model validates the present observations that in most embryos, the human ICM is clonally imbalanced and largely populated by one 2-cell clone. Overall, the model predicts that modulation of cell death, cell arrest and ACD result in a distribution of embryos that will have clonally imbalanced ICMs.

### Example 7

Lineage Tracing of Time-Lapse Movies at the 8-to-16-Cell Division in Human Embryos

To extend the observations of ACD from live imaging (FIGS. 5A-5G), multifocal, time-course brightfield movies of human embryos developing from zygote to the blastocyst stage in the IVF clinic with Embryoscope™ were analyzed (FIG. 7A and FIG. 14, panel A, Example 1). Multifocal image quality was sufficient to manually track the lineage history of 54 embryos (Methods). This resulted in a lineage tree for each embryo, in which the timing of division of each blastomere at 2-, 4- and 8-cell stage, as well as the clonal origin of each cell were registered.

Similar to the live imaging analysis using dyes (FIGS. 4A-4F), 8-cell stage blastomere divisions were scored as SCD if they resulted in two daughter cells that remained outside, or as ACD if one of the daughters ingressed to form the emerging ICM (FIG. 7B and FIG. 14, panel B). In most of the 8-cell embryos analyzed (53/54; 98.1%) between one and three ACDs were observed, and one embryo in which four cells had ACDs (FIG. 7C). These numbers are consistent with the previous observations (FIGS. 5C-D) and the number of ACDs used in the statistical model (FIG. 13, panels E-G). Interestingly, these numbers are lower than those in mice at the same stage, which show 2.84 ACD/embryo on average at the 8- to 16-cell transition. In contrast, human embryos had a lower average of 1.96 ACD/embryo at the same stage.

In 8-cell embryos with one ACD (16/54 embryos, 29.6%) or three ACDs (12/54, 22.2%), the ICM composition at the 16-cell stage was, by definition, clonally imbalanced. In 3.7% of embryos (2/54), all three ICM cells originated from the same 2-cell clone (FIG. 7E). In embryos with two ACDs (25/54, 46.3%), ˜44% of embryos (11/25) had ICM cells that originated from the same 2-cell blastomere and had a clonally imbalanced ICM (FIG. 7D). The distribution of ACDs measured in the whole population revealed a majority of embryos (39/54, 72.2%) with unbalanced ICM founder cell allocation at the 16-cell stage (FIG. 7F). Notably, 29/54 (53.7%) 16-cell stage human embryos had inside cells that were derived completely from just one of the two 2-cell blastomeres.

It was considered that successive rounds of ACDs at the 16- to 32- and 32- to 64-cell transitions could potentially restore clonal balance to the ICM. Cell compaction at the 16-cell stage (after which individual blastomeres could not be accurately followed in the Embryoscope™ movies) precluded us from tracing cells further. The mathematical model incorporates this possibility, so it was used to project how different “starting” degrees of clonal imbalance at the 16-cell stage would be predicted to affect the composition of the ICM at the blastocyst stage. Interestingly, early clonal imbalances in ICM founder cells were largely inherited to the blastocyst stage, with only a ˜12% chance for the original imbalance to be either neutralized or reversed (FIG. 7G). Also, the clonal composition of the embryos imaged by Embryoscope™ (projected at the blastocyst stage) was statistically consistent with the clonal distributions measured in the TE and ICM of the embryos labelled at the 2-cell stage and analyzed at the blastocyst stage (FIG. 14, panel C). These results cross-validate the original observations with an independent dataset. Together, these data indicate that a low number of ACDs at the 8- to 16-cell transition in human embryos leads in a large clonal asymmetry in the ICM.

### Example 8

### The Faster Dividing 2-Cell Stage Blastomere is Biased Towards the First Asymmetric Division at the 8-Cell Stage

In mice, the 2-cell stage blastomere that divides first is reported to contribute more cells to the ICM and polar TE, whereas the blastomere that divides later gives rise to more cells of the mural TE. This example aimed to evaluate whether such a connection between division asynchrony at the 2-cell stage and ACD at the 8- to 16-cell transition exists in human embryo development.

Most embryos analyzed (46/54, 85.2%) exhibited asynchronous divisions at the 2- to 4-cell transition, with one blastomere (labelled “A”) dividing 15-120 min faster than the other (labelled “B”, FIG. 7H). Division asynchrony was inherited through to the 4- and 8-cell stages, with daughter cells of the faster blastomere also dividing faster at these stages (FIGS. 7I-K).

To investigate the relationship between clonal identity, division time, and ACD, asynchronous embryos were examined (FIG. 7L). In 31/46 embryos (67.4%), the first ACD originated from the faster “A” 2-cell blastomere (FIG. 7M). In embryos with more than 1 ACD, the second ACD was mostly from the “B” blastomere (21/31; 67.7%). In embryos with 3 ACD, the third ACD was by the “A” blastomere in 6/10 embryos (FIG. 7N).

In total, 43.5% of the asynchronous embryos analyzed (20/46) contained more “A” cells than “B” (FIG. 7O), whereas only 28.3% of embryos (13/46) contained more “B” cells than “A” cells in their ICM (FIG. 7O). In the remaining 13/46 (28.3%) embryos, ICM founders at the 16-cell stage were clonally balanced, with as many “A” cells as “B” (FIG. 7O). These results indicate that the blastomere that divides first at the 2-cell stage is biased to contribute more ICM founder cells at the 16-cell stage (FIG. 7P, FIG. 14, panel D).

The mathematical model was used to determine if this bias would affect the clonal composition of the blastocyst. A fourth parameter that “biased” one 2-cell clone towards having more ACDs was applied. Blastomeres were still randomly selected for internalization at the same total numbers, but with unequal probability for GFP+ and GFP− clones. It was found that a lineage bias of between 50% and 80% resulted in a model that was statistically consistent with the observed data (FIG. 6G).

The simulations were repeated using a 70% bias and compared the clonal imbalances in the predicted embryos with the observations in the real human embryos. The percentage of GFP+ cells in the whole blastocyst, the size of the ICM and the clonal distributions in the TE and ICM in the predicted embryos showed statistical agreement with the data from the real embryos (FIGS. 6B-E). Importantly, the 70% bias in this model improved the inheritance of clonal imbalances in the ICM from the 16-cell stage to the blastocyst stage (FIG. 7G) and the fraction of embryos with a clonally imbalanced TE or ICM more closely mimicked the observed data (FIG. 6F).

Overall, the data presented herein indicate that the blastomere that divides faster at the 2- to 4-cell transition will generate more of the very few asymmetrically dividing cells in the initial wave and contribute more cells to the ICM in the human blastocyst.

### ADDITIONAL CONSIDERATION

After fertilization, the zygote divides to generate cells that are thought to remain equivalent to each other until the first fate diversification event. This delayed fate specification would predict that each cell of the 2-cell embryo gives rise to, on average, half of all the cells in human bodies. However, genome sequence and single nucleotide polymorphism-based retrospective lineage reconstruction of human development has suggested clonal imbalances in the human body, such that one cell from the two-cell embryo is often dominant. What is the reason behind this imbalance is not known. Indeed, when and how cell fate decisions are initiated in the human embryo remain long standing questions, because the access to human embryos for research is extremely limited.

During embryo development, the first three fates are specified by three successive waves of asymmetric cell divisions that position one daughter cell inside the embryo (inner cell mass, ICM) and the other on the outside. Cells positioned to the outside generate trophectoderm (TE, future placenta). Intriguingly, in mouse embryos, cells internalized during the first wave (8- to 16-cell stage) contribute mainly to epiblast (future body) whereas those internalized during the second (16- to 32-cell stage) and third waves (32- to 64-cell stage) tend to generate primitive endoderm (hypoblast in human, future yolk sac). Therefore, the number and timing of internalized cells influences the clonal composition of the future mouse body. Whether successive specification of the inner cells happens in human embryos is not known.

Although both blastomeres of the 2-cell mouse embryo, and most blastomeres in 4-cell embryos, contribute to both ICM and TE, lineage tracing and single cell RNA sequencing studies suggest that the fate and developmental potential of mouse blastomeres at the 2-cell and 4-cell stages are unequal. Molecular asymmetries in gene expression and epigenetic modifications at the 2-cell and 4-cell stages were found to contribute to their fate. Importantly, the primary bias of mouse blastomeres is compatible with developmental plasticity; the two can co-exist.

To determine how early blastomeres contribute to the three lineages of the human embryo, lineage tracing of live human embryos from the first cleavage division until the blastocyst stage was performed. The present disclosure discovered that most cells of the epiblast, the future human body, originate from just one of the two cells. Notably, the first blastomere to divide at the 2-cell stage has a higher likelihood to generate the first and more internalized cells at the 8- to 16-cell stage. The limited number of cell internalizations at the first wave of asymmetric cell divisions in the human embryo can be a bottleneck that creates a large clonal imbalance in the ICM. The present data indicate an interplay between cell division dynamics in the early embryo lead to clonal asymmetries in the human body.

In the present disclosure, prospective lineage tracing, imaging of live human embryos, and mathematical modeling were used to track the fate of each of the 2-cell human blastomeres for the first time. It is demonstrated herein that human 2-cell blastomeres contribute unequally to the ICM and polar TE, with one blastomere contributing a majority of epiblast cells that will form the body. Importantly, the results show that this clonal imbalance is linked with the first blastomere to divide in the 2-cell human embryo being biased to contribute cells undertaking more asymmetric divisions, generating the small number of founding epiblast cells.

On average 71.25% of ICM cells originate from one 2-cell blastomere: while in most embryos (19 out of 22) one 2-cell blastomere contributed ≥60% of ICM cells, the 2-cell clonal contributions to the ICM ranged from near equal (˜50% for 3/22 embryos) to fully biased (100% for 2/22 embryos). In contrast, the 2-cell clonal composition of the TE is more balanced, with approximately half the embryos analyzed containing comparable numbers of cells from each 2-cell clone in their TE. These findings provide embryological validation of previous results suggesting a universal unequal contribution of early embryonic cells to the human body. Remarkably, the range of clonal imbalance reported using multiple approaches matches the one predicted in these publications.

The results support the hypothesis that clonal imbalances established during the earliest stages of human development affect the final lineage composition even though the precise lineage commitment of the 2-cell blastomeres is not deterministic. Imbalances in clonal composition can arise when ICM first forms at the 8- to 16-cell division. An uneven number of ACDs, or an even number of ACDs that have the same clonal identity, by definition generate a clonal imbalance. Importantly, however, the extent of the clonal imbalance decreases with the number of ACDs: 1 ACD will always generate a 100% imbalance, 2 ACD will generate a 100% bias close to half of the time, 3 ACD will generate a 100% imbalance 16% of the time, etc. Together with the previous work, a substantially lower number of ACDs during the 8- to 16-cell stage division was observe in human compared to mouse embryos. In mouse embryos dividing from 8- to 16-cell stage, 21% had 1 ACD; 26%, 2 ACD; 21%, 3 ACD; 11%, 4 ACD; and 21%, 5 ACD. In contrast, in human, during a similar transition it was found that 30% had 1 ACD; 46%, 2 ACD; 22%, 3 ACD; and 2%, 4 ACD. Others have also noted that the number of inner cells at the morula stage, and the ICM size is larger in mouse than in human embryos⁴⁹. This lower distribution of ACDs during the 8- to 16-cell division in human embryos anticipates a high likelihood of a clonal imbalance when compared to mouse. It will be of interest to decipher how the number of ACDs is regulated in different mammalian species and the impact on development and clonal composition of the body.

The present study also allowed investigation of how the topographical distribution of clones in the TE connects with asymmetries in composition of the ICM. 2-cell clones were organized in coherent TE cell clusters, indicating little cell mixing at the 8- to 32-cell stages, as in the mouse¹⁵. In mouse embryos, zygotic division along the animal-vegetal axis results in one 2-cell blastomere showing a biased contribution to the cells of the embryonic half of the embryo (the epiblast and the overlaying polar TE), and the other forming the abembryonic mural TE^{12,50}. However, in humans, the largest cell cluster for each clone spread along the embryonic-abembryonic axis, resulting in the presence of cells from both clones in the polar TE. Despite this, and similarly to the mouse embryo, the 2-cell clonal composition in the human polar TE correlates with the clonal composition of the epiblast. Differences and similarities in the mechanisms that self-organize human and mouse embryos include differences in the temporal sequence of pre-implantation morphogenetic events, such as cell differentiation, polarization, compaction, and cavitation.

Genomic instability and aneuploidy have been suggested as drivers of asymmetric 2-cell clonal contribution in the human embryo. Most embryos used in this research were euploid, and the few aneuploid embryos present exhibited ranging 2-cell clonal bias to the ICM and the TE. This suggests not only that genomic instabilities were not a primary cause for biased lineage allocations, but also that the dataset used herein was composed of healthy human embryos. Moreover, a minority of embryos that implant and give rise to healthy births contain aneuploid cells, and most aneuploid embryos are postulated to be lost during pregnancy. Therefore, the asymmetric clonal distributions detected in human adults are likely not a result of embryonic aneuploidy. This is further supported by the lineage tracing of embryos imaged with Embryoscope™, which recorded embryos that ultimately gave rise to a heathy birth while also exhibited clonal imbalances in their founding ICM population at the 16-cell stage. The data infer that imbalanced lineage allocation reflect intrinsic developmental trajectories occurring in developing human embryos.

Several reports suggest that biased cell fate allocation in mouse embryos traces back to the cleavage division of the zygote, resulting in asymmetries in the 2-cell embryo that are maintained and amplified in further cleavage divisions. At the 8-cell stage, the cells derived from one of these two mouse blastomeres have a higher number of asymmetric cell divisions and thus make a greater contribution to the ICM. The present lineage tracing of 54 heathy human embryos from the 2- to 16-cell stage revealed that the first 2-cell blastomere to divide had a higher propensity for the first ACD in its descendants. In these 16-cell human embryos, only 28% had a clonally balanced population of inside cells. The faster dividing 2-cell blastomere dominated the population in 61% of the remaining embryos. Although mathematical modelling demonstrates that the observed clonal imbalance of the ICM can emerge from blastomeres that randomly divide asymmetrically, it indicates that a lineage bias for ACD remains statistically plausible up to 80:20 (versus an unbiased propensity for ACD). To what extent 2-cell asymmetries impact the clonal imbalance will require additional research and the present results indicate cooperation between stochastic and biasing mechanisms controlling the clonal makeup of the human embryo.

As described herein, the human blastocyst is clonally imbalanced, with most of the epiblast cells, and therefore the future body, originating from only one of the blastomeres at the 2-cell stage. The data provided herein show that early bottlenecks of few ACDs at the 8-cell embryo stage lead to overrepresentation of descendants of one 2-cell blastomere in the ICM. Consequently, the human bodies are derived mostly from a single 2-cell blastomere. Moreover, the present data suggest that asynchronous cell division of the 2-cell blastomeres influence the ultimate lineage composition.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for determining a clonal composition of an embryo, comprising:
culturing an embryo at the zygote stage in a first embryo culture media until the embryo forms 2-cell blastomeres;
labeling one blastomere of the 2-cell blastomeres with a detectable lineage marker;
culturing the 2-cell blastomeres in a second embryo culture media for about 4 to 5 days allowing the 2-cell blastomeres to develop into a blastocyst;
detecting cells expressing the detectable lineage marker in the blastocyst; and
quantifying the clonal composition of the inner cell mass (ICM) and trophectoderm (TE) based on the detection of cells expressing the detectable lineage marker.

2. (canceled)

3. The method of claim 1, wherein the first embryo culture media and/or the second embryo culture media comprises amino acids, physiological salts, a carbon source, an antibiotic, and a buffer, wherein the carbon source is glucose.

4.-9. (canceled)

10. The method of claim 1, wherein the embryo at the zygote stage is cultured in the first embryo culture media for about 12-20 hours until the completion of the first cleavage division.

11. The method of claim 1, (i) wherein the detectable lineage marker does not affect the development of the embryo to the blastocyste stage and enables annotation of the position and boundaries of cells in the embryo; (ii) wherein labeling the one blastomere of the 2-cell blastomeres with the detectable lineage marker comprises injecting the blastomere with an mRNA encoding the detectable lineage marker; and/or (iii) wherein the blastocyst is a non-expanded blastocyst or an expanded blastocyst.

12. (canceled)

13. (canceled)

14. The method of claim 1, further comprising selecting a subset of embryos at 4-cell stage, 8-cell stage, 16-cell stage, and/or 32-cell stage from the second embryo culture media prior to the formation of the blastocyst, and live staining the subset of embryos, wherein live staining the subset of embryos comprises culturing the selected subset of embryos in an embryo culture media containing dyes and wherein the dyes are membrane-permeable fluorescent dyes capable of tracking both genomic nucleic acids and a component of cytoskeleton of the embryos.

15.-21. (canceled)

22. The method of claim 1, wherein quantifying the clonal composition of the ICM and TE further comprises (i) identifying the dominant clonal composition in the ICM and/or the TE of the blastocyst; and/or (ii) determining the percentage of cells expressing the detectable lineage marker and/or cells not expressing the detectable lineage marker in the ICM and/or TE of the blastocyst.

23.-25. (canceled)

26. The method of claim 1, wherein the embryo is a human embryo.

27. A method of selecting embryos, comprising
providing a plurality of embryos at the zygote stage;
determining a clonal composition of each embryo of the plurality of embryos according to claim 1; and
selecting embryos having a desired clonal composition based on the percentage of cells expressing the detectable lineage marker and/or cells not expressing the detectable lineage marker in the inner cell mass (ICM) and trophectoderm (TE) of an embryo at the blastocyst stage.

28. The method of claim 27, wherein the selected embryo comprises clonally imbalanced ICM.

29. (canceled)

30. The method of claim 27, wherein the embryos are human embryos.

31. A computer-based method of determining a clonal composition in embryo models, comprising:
(i) generating a plurality of embryo models each comprising two cells, wherein one cell of each embryo model is randomly marked;
(ii) modulating a set of parameters comprising a cell death rate, a cell arrest rate, and a number of asymmetric cell divisions for a stochastic model;
(iii) subjecting the plurality of embryo models to the stochastic model wherein each embryo model undergoes successive rounds of cell division until the embryo model reaches a desired total number of cells; and
(iv) determining a clonal composition of the inner cell mass (ICM) and trophectoderm (TE) for each embryo model reaching the desired total number of cells.

32. (canceled)

33. (canceled)

34. The method of claim 31, wherein (i) each embryo model undergoes at least five rounds of cell division; and/or (ii) the desired total number of cells is at least 64.

35. (canceled)

36. The method of claim 31, wherein modulating the set of parameters comprises (i) selecting the number of asymmetric cell divisions for the 8- to 16-cell transition, 16- to 32-cell transition, and/or 32- to 64-cell transition, optionally, the number of asymmetric cell divisions is selected as 0, 1, 2, or 3; (ii) selecting the cell death rate for cell divisions beyond the 64-cell stage; and/or (iii) selecting the cell arrest rate at the 4-cell stage and/or the 8-cell stage.

37.-45. (canceled)

46. The method of claim 31, wherein modulating the set of parameters comprises fitting the set of parameters to in vitro clonal composition data.

47. The method of claim 46, further comprising providing the in vitro clonal composition data.

48. (canceled)

49. The method of claim 31, wherein the determined clonal composition comprises the percentage of marked cells and/or unmarked cells in the ICM and/or TE of each embryo model; and/or wherein the plurality of embryo models is a plurality of human embryo models.

50. (canceled)

51. A method for investigating the effect of a test agent on embryonic development, comprising:
contacting a test agent with an embryo at the zygote stage;
determining a clonal composition of the embryo according to claim 1; and
determining the effect of the test agent on the clonal composition, optionally the determining comprises comparing the clonal composition obtained in the presence of the test agent with a clonal composition obtained in the absence of the test agent; and optionally wherein the embryo is a human embryo.
